(19) United States
(12) Patent Application Publication
(10) Pub. No.: US 2023/0193269 A1
(43) Pub. Date: Jun. 22, 2023

(54) OLIGONUCLEOTIDES FOR SPLICE MODULATION OF CARD9

(71) Applicant: BOEHRINGER INGELHEIM INTERNATIONAL GMBH, Ingelheim am Rhein (DE)

(72) Inventors: Peter HAGEDORN, Horsholm (DK); Lars JOENSON, Horsholm (DK); Anja Moehart HOEG, Horsholm (DK); Jonas VIKESSA, Horsholm (DK); Fergus BYRNE, Ridgefield, CT (US); Jay FINE, Ridgefield, CT (US); Mouhamadou MBOW, Ridgefield, CT (US); Joe Adam WAHLE, Ridgefield, CT (US); Elliott Sanford KLEIN, Ridgefield, CT (US); Kristina Mary SAI, Ridgefield, CT (US); Fei SHEN, Ridgefield, CT (US)

(21) Appl. No.: 17/999,148

(22) PCT Filed: May 22, 2020
(86) PCT No.: PCT/EP2020/064285

§ 371 Date: Nov. 17, 2022

(51) Int. Cl. C12N 15/113; A61K 31/7088

(52) U.S. Cl. CPC C12N 15/113; A61K 31/7088; C12N 2310/11; C12N 2310/3231; C12N 2320/33

(57) ABSTRACT

The present invention relates to splice modulating oligonucleotides (oligomers) that are complementary to the CARD9 pre-mRNA, for inhibiting the inclusion of a CARD9 exon 11 in CARD9 mRNA. The oligonucleotides of the invention are useful in the treatment of inflammatory diseases such as IBD.

### REFERENCE TO SEQUENCE LISTING

The content of the electronically-submitted sequence listing (Name: P35120_Sequence_listing_project_ST25.txt; Size: 130,099 bytes; and Date of Creation: May 5, 2020) submitted in this application is herein incorporated by reference in its entirety.

### FIELD OF INVENTION

The present invention relates to splice modulating oligonucleotides (oligomers) that are complementary to the CARD9 pre-mRNA, which reduce the incorporation of a CARD9 exon 11 in the mature CARD9 mRNA. The oligonucleotides of the invention are useful in the treatment of a range of medical disorders, including inflammatory diseases such as inflammatory bowel disease (such as Crohn’s disease or ulcerative colitis); or a disease selected from the group consisting of pancreatitis, IgA nephropathy, primary sclerosing cholangitis, cardiovascular disease, cancer and diabetes.

### BACKGROUND

CARD9 (Caspase recruitment domain-containing protein 9) is a central component of anti-fungal innate immune signaling via C-type lectin receptors. It is a member of the CARD family which plays an important role in innate immune response by the activation of NF-_{K}B. CARD9 mediates pro-inflammatory cytokine production, including TNFα, IL-6, and IL-1β, thereby regulating the responses of Th1 and Th17 cells.

CARD9 has been associated with many diseases and disorders. For example, CARD9 expression has been associated with cardiovascular disease, autoimmune disease, cancer and obesity (Zhong et al. Cell Death and Disease (2018) 9:52).

Further, CARD9 has been identified as a gene associated with the risk of inflammatory bowel disease (IBD), ankylosing spondylitis, primary sclerosing cholangitis, and IgA nephropathy (Cao et al., Immunity 2015 Oct 20; 43(4): 715-726).

Yamamoto-Furusho showed that expression of CARD9 can differently distinguish active and remission ulcerative colitis (UC). Therefore, CARD9 was proposed as target for in UC patients (Journal of Inflammation (2018) 15:13).

Further, it was shown that CARD9 expression is upregulated in severe acute pancreatitis (SAP) patients. Small interfering RNAs (siRNAs) were used to reduce the levels of CARD9 expression in sodium taurocholate-stimulated SAP rats. When compared to the untreated group, the cohort that received the siRNA treatment demonstrated a significant reduction in pancreatic injury, neutrophil infiltration, myeloperoxidase activity and pro-inflammatory cytokines. Therefore, CARD9 was suggested as target for the treatment of acute pancreatitis (Yang et al., J Cell Mol Med. 2016;21(6):1085-1093).

Moreover, CARD9 was proposed as target for the treatment of neutrophilic dermatoses (Tartey et al., The Journal of Immunology Sep. 15, 2018, 201 (6) 1639-1644).

Unlike most genetic risk factors for complex diseases, CARD9 alleles exist in both predisposing and protective forms for IBD. The predisposing variant, CARD9 S12N, is a common coding single nucleotide polymorphism that was identified via genome-wide association studies (GWAS) and is associated with increased expression of CARD9 mRNA (Cao et al., Immunity 2015 Oct 20; 43(4): 715-726). The protective variant, CARD9 S12NΔ11, is a rare splice variant in which exon 11 of CARD9 is deleted. This allele, identified by deep sequencing of GWAS loci, results in a protein with a C-terminal truncation and confers strong protection against disease (P < 10-16) (Beaudoin et al., PLoS Genet. 2013;9:e1003723; Rivas et al., Nat Genet. 2011;43:1066-1073).

Small molecule inhibitors have been used to directly target the CARD9 exon 11 splicing event to mimic the protective CARD9 S12NΔ11 variant to determine the feasibility of using small using small-molecule inhibitors to recapitulate the anti-inflammatory function of CARD9 mutations associated with protection from IBD (Leshchiner et al., Proc Natl Acad Sci USA. 2017 Oct 24; 114(43): 11392-11397).

Antisense-mediated splicing modulation is a tool that can be exploited in several ways to provide a potential therapy for rare genetic diseases. This approach has resulted in approved therapeutics for Duchenne muscular dystrophy and spinal muscular atrophy - see Arechavala-Gomeza et al., Appl Clin Genet. 2014; 7: 245-252 for a review of various approaches for splice modulation using antisense oligonucleotides.

The present inventors have developed splice switching compounds targeting a CARD9 exon 11. The compounds of the invention modulate the inclusion of the CARD9 exon 11 in the CARD9 mRNA, thereby enhancing the expression of a dΔ11 CARD9 mRNA.

### OBJECTIVE OF THE INVENTION

The present invention provides splice modulating oligonucleotides (oligomers) that are complementary to the CARD9 pre-mRNA, which are capable of modulating the inclusion of the CARD9 exon 11, thereby enhancing the expression of CARD 9 dΔ11 variant. CARD 9 dΔ11 protein variant is a CARD9 variant which has been illustrated to provide protection from inflammatory bowel diseases. Alternatively stated, the oligonucleotides of the invention are splice switching oligonucleotides which result in the skipping of exon1 1 in the processing of the CARD9 pre-mRNA, resulting in a mature mRNA which lacks part of or all of the CARD9 exon 11 sequence. The expression of the resulting CARD 9 dΔ11 transcript results in the expression of, or increased expression of the CARD 9 dΔ11 protein.

The oligonucleotides of the invention are useful in the treatment of a range of medical disorders as disclosed herein, such as inflammatory bowel disease (such as Crohn’s disease and ulcerative colitis); or a disease selected from the group consisting of pancreatitis, IgA nephropathy, primary sclerosing cholangitis, cardiovascular disease, cancer and diabetes

### SUMMARY OF THE INVENTION

The present invention provides antisense oligonucleotides which are complementary to, and which are capable of modulating the expression of a CARD9 nucleic acid, and for their use in medicine.

The present invention relates to the identification of advantageous target site sequences present within a CARD9 nucleic acid, which are suitable for targeting with antisense oligonucleotides, notable target sites which result in the modulation of the splicing of the CARD9 pre-mRNA.

Advantageously, the oligonucleotides of the invention are capable of enhancing the skipping of exon1 1 in the processing of the CARD9 pre-mRNA, resulting in a mature mRNA which lacks part of or all of the CARD9 exon 11 sequence. The expression of the resulting CARD 9 dΔ11 transcript results in the expression of, or increased expression of the CARD 9 dΔ11 protein (CARD9 Δ11), a protein variant which provides protection or alleviation of inflammatory conditions, such as inflammatory bowel disease.

The present invention provides splice modulating oligonucleotides (oligomers) that are complementary to the CARD9 pre-mRNA which reduce the expression of the wildtype CARD9 (WTCARD9) protein. Reduction of the WT CARD9 protein may be achieved by the production of an alternative transcript, such as the CARD9Δ11 mRNA. In some embodiments, the reduction in expression of WTCARD9 may be due to transcript de-stabilization (e.g. via non-sense-mediated decay).

The invention further provides antisense oligonucleotides for use in the prevention or the treatment of a disease selected from the group consisting of inflammatory bowel disease (such as Crohn’s disease and ulcerative colitis), pancreatitis, IgA nephropathy, primary sclerosing cholangitis, cardiovascular disease, cancer and diabetes.

The invention further provides antisense oligonucleotides for use in the prevention or the treatment of an inflammatory disorder such as inflammatory bowel disease (such as Crohn’s disease and ulcerative colitis).

Advantageously, the oligonucleotides of the invention are capable of modulating the splicing on the CARD9 pre-mRNA so as to inhibit the inclusion of an exon 11 sequence of the CARD9 pre-mRNA (e.g. SEQ ID NO 1).

The inventors have identified a region of the human CARD9 pre-mRNA (illustrated herein as SEQ ID NO 1), which is particularly sensitive to targeting with antisense oligonucleotides in order to modulate the splicing of exon 11 of the CARD 9 pre-mRNA, for example the target sites identified as SEQ ID NO 6, 7, 8, 9, 559, 560, 561, 562, 563, 564 or 565 herein.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as between about 10 - and about 40 nucleotides in length, which are complementary to target sites, such as fully complementary to SEQ ID NO 6.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to, including, in some cases, fully complementary, to SEQ ID NO 7.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 8.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 9.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 559.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 560.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 561.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 562.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 563.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 564.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 40 nucleotides in length, which are complementary to such as fully complementary to SEQ ID NO 565.

In some embodiments, the contiguous nucleotide sequence is 10 - 25 nucleotides in length, or 10 - 20 nucleotides in length which is fully complementary to the target sequence, such as a target sequence selected from the group consisting of SEQ ID NO 1, 6, 7, 8, 9, 559, 560, 561, 562, 563, 564 or 565.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 6.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 7.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 8.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 9.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 559.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 560.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 561.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 562.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 563.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 564.

The invention provides antisense oligonucleotides which are capable of modulating the splicing of the CARD9 pre-mRNA, wherein the antisense oligonucleotide comprises a contiguous nucleotide sequence of at least 10 nucleotides in length, such as 10 - 20 nucleotides in length, which are complementary to such as fully complementary to SEQ ID NO 565.

The invention provides antisense oligonucleotides which are capable of modulating the splicing of the CARD9 pre-mRNA, wherein the antisense oligonucleotide comprises a contiguous nucleotide sequence of at least 10 nucleotides in length, which is complementary to such as fully complementary to a sequence selected from the group consisting of SEQ ID NO 11 - 284.

The invention provides antisense oligonucleotides which are capable of modulating the splicing of the CARD9 pre-mRNA, wherein the antisense oligonucleotide comprises a contiguous nucleotide sequence of at least 12 nucleotides in length, which is complementary to such as fully complementary to a sequence selected from the group consisting of SEQ ID NO 11 - 284.

The invention provides antisense oligonucleotides which are capable of modulating the splicing of the CARD9 pre-mRNA, wherein the antisense oligonucleotide comprises a contiguous nucleotide sequence of at least 14 nucleotides in length, which is complementary to such as fully complementary to a sequence selected from the group consisting of SEQ ID NO 11 - 284.

The invention provides antisense oligonucleotides which are capable of modulating the splicing of the CARD9 pre-mRNA, wherein the antisense oligonucleotide comprises a contiguous nucleotide sequence of at least 10 nucleotides which are identical to a region of a sequence selected from SEQ ID NO 285 - 558.

The invention provides antisense oligonucleotides which are capable of modulating the splicing of the CARD9 pre-mRNA, wherein the antisense oligonucleotide comprises a contiguous nucleotide sequence of at least 10 nucleotides present in a sequence selected from SEQ ID NO 285 - 558.

The invention provides antisense oligonucleotides which are capable of modulating the splicing of the CARD9 pre-mRNA, wherein the antisense oligonucleotide comprises a contiguous nucleotide sequence which is identical a sequence selected from SEQ ID NO 285 - 558.

The invention provides antisense oligonucleotides which are capable of modulating the splicing of the CARD9 pre-mRNA, wherein the antisense oligonucleotide comprises a contiguous nucleotide sequence selected from SEQ ID NO 285 - 558.

The invention provides an antisense oligonucleotide selected from the group consisting of compound ID No #285 - #558. The compounds are shown in the “Compound and Data Table” in the Examples section (see column “Oligo_structure”).

The oligonucleotide of the invention may comprise one or more conjugate groups, i.e. the oligonucleotide may be an antisense oligonucleotide conjugate.

The invention provides pharmaceutical compositions comprising the antisense oligonucleotide of the invention and a pharmaceutically acceptable diluent, carrier, salt and/or adjuvant.

The invention provides for a pharmaceutically acceptable salt of the antisense oligonucleotide of the invention. In some embodiments, the pharmaceutically acceptable salt is a sodium salt, a potassium salt or an ammonium salt.

The invention provides for a pharmaceutical solution of the oligonucleotide of the invention, wherein the pharmaceutical solution comprises the oligonucleotide of the invention and a pharmaceutically acceptable solvent, such as phosphate buffered saline.

The invention provides for the oligonucleotide of the invention in solid powdered form, such as in the form of a lyophilized powder.

Typically, the antisense oligonucleotide of the invention comprises a contiguous nucleotide sequence of at least 10 nucleotides in length, such as 10 - 30 nucleotides in length, wherein the contiguous nucleotide sequence is at least 90% complementary to, such as fully complementary to, the CARD9 target nucleic acid, such as SEQ ID NO 1, 6, 7, 8, 9, 559, 560, 561, 562, 563, 564 or 565. In some embodiments, all of the nucleosides of the antisense oligonucleotide form the contiguous nucleotide sequence.

In some embodiments, the antisense oligonucleotide of the invention has a length of up to 30 nucleotides, and comprises a contiguous nucleotide sequence of at least 10 nucleotides in length, such as 10 - 30 nucleotides in length, wherein the contiguous nucleotide sequence is at least 90% complementary to, such as fully complementary to, the CARD9 target nucleic acid, such as SEQ ID NO 1, or a target site therein such as a target site (target sequence) selected from the group consisting of SEQ ID NO 6, 7, 8, 9, 559, 560, 561, 562, 563, 564 or 565.

Advantageously, the antisense oligonucleotide of the invention is capable of modulating the splicing of the human CARD9 pre-mRNA, such as to inhibit or reduce the inclusion of exon 11 CARD9 pre-mRNA in the CARD9 mRNA. This results in the expression of a Δ11CARD9 variant (as illustrated herein as the amino acid sequence provided as SEQ ID NO 5).

The invention provides an in vivo or in vitro method for modulating the splicing of a mammalian CARD9 pre-mRNA in a target cell which is expressing a CARD9 pre-mRNA, by administering an oligonucleotide or composition of the invention in an effective amount to said cell, wherein the administration of the oligonucleotide results in the expression, or an increase in expression, of a CARD9Δ11 mRNA in the cell (for illustration as shown in SEQ ID NO 4). The expression of the CARD9Δ11 mRNA in the cell results in the expression or enhanced expression of the CARD9 Δ11 protein (as illustrated herein as the amino acid sequence provided as SEQ ID NO 5). As described elsewhere herein, expression of the CARD9Δ11 mRNA or protein may be increased as compared to untreated control cells.

In some embodiments, the administration of the oligonucleotide or composition of the invention results in a decrease in expression of the CARD9 WT mRNA (SEQ ID NO 2) or protein (for illustration see SEQ ID NO 3). In some embodiments the administration of the oligonucleotide or composition of the invention results in a change in ratio of alternative splice variants of the CARD9 mRNA, for example an increase in the ratio of expression of CARD9Δ11 mRNA compared to the WTCARD9 mRNA (i.e. CARD9 mRNA which comprises the CARD9 exon 11 sequence).

Modulation of CARD9 expression may therefore refer to a change in the ratio of splice variant transcripts of CARD9, such as an decrease in the level of the WTCARD9 mRNA (such as illustrated by SEQ ID NO 2), and/ or an increase in the level of CARD9 Δ11 mRNA (such as illustrated by SEQ ID NO 4). In some embodiments, the ratio of the level of CARD9 Δ11 mRNA to the level of WTCARD9 mRNA comprising exon 11 is increased. Accordingly, the oligonucleotides of the present invention, when present in a target cell, shall be capable of increasing the aforementioned ratio.

Modulation of CARD9 expression may also refer to a change in the ratio of proteins encoded by splice variants transcripts, such as an decrease in the level of the WTCARD9 protein (such as illustrated by SEQ ID NO 3), and/ or an increase in the level of CARD9 Δ11 protein (such as illustrated by SEQ ID NO 5). In some embodiments, the ratio of the level of CARD9 Δ11 protein to the level of WTCARD9 protein is increased. Accordingly, the oligonucleotides of the present invention, when present in a target cell, shall be capable of increasing the aforementioned ratio.

Splice modulating oligonucleotides typically operate via an occupation based mechanism rather than via a degradation mechanism (such as RNaseH mediated inhibition). However, splice modulation can result in the alternative transcript being degraded, e.g. via non-sense mediated decay.

### SEQUENCE LISTING

The sequence listing submitted with this application is hereby incorporated by reference. In the event of a discrepancy between the sequence listing and the specification or figures, the information disclosed in the specification (including the figures) shall be deemed to be correct. It will be understood that in reference to target nucleic acids or target sequences or target sites, the sequences disclosed herein refer to DNA sequences derived from genomic or cDNA sequences, and are provided as representations of the nucleic acids in the cell, in vitro or in vivo, which may for instance be RNA molecules (for instance in the RNA target sequences uracil (U) is present in place of the thymine (T) shown in the enclosed DNA sequences). Target nucleic acids such as SEQ ID NO 6 to 9, 11 to 284, 559 to 565 include RNA sequences transcribed from the reference target sequence (such as SEQ ID NO 1 or naturally occurring variant thereof).

CARD9 Reference Sequences

| | |
|---|---|
| SEQ ID NO 1 | Homo sapiens CARD9 pre-mRNA reference sequence |
| SEQ ID NO 2 | Homo sapiens CARD9 mRNA reference sequence (WT) |
| SEQ ID NO 3 | Homo sapiens CARD9 protein reference sequence (WT) |
| SEQ ID NO 4 | Homo sapiens CARD9 Δ11mRNA reference sequence (Δ11) |
| SEQ ID NO 5 | Homo sapiens CARD9 Δ11 protein reference sequence (Δ11) |
| SEQ ID NO 6 | Homo sapiens CARD9 - Δ11 target region |
| SEQ ID NO 7 | Homo sapiens CARD9 - Δ11 target sub-region 1 |
| SEQ ID NO 8 | Homo sapiens CARD9 - Δ11 target sub-region 2 |
| SEQ ID NO 9 | Homo sapiens CARD9 - Δ11 target sub-region 3 |
| SEQ ID NO 10 | Amino acid sequence encoded by CARD9 exon 11 - illustrative sequence CLAGGGSPKQPFAALHQEQVLRNPH |
| SEQ ID NO 11 - 284 | Homo sapiens CARD9 - Δ11 target sites |
| SEQ ID NO 285 - 558 | Oligonucleotide sequence motifs |
| Compounds #285 - #558 | Exemplified compounds |
| SEQ ID NO 559 | Homo sapiens CARD9 - further Δ11 target sub-region: ACTGGACCCATGTGGGGATC |
| SEQ ID NO 560 | Homo sapiens CARD9 - further Δ11 target sub-region: GCACGACTCTCCTTTCCAGGCTGCCTTGCCGG |
| SEQ ID NO 561 | Homo sapiens CARD9 - further Δ11 target sub-region: AGGCTGCCTTGCCGGCGGGGGGAGCCCGAAAC |
| SEQ ID NO 562 | Homo sapiens CARD9 - further Δ11 target sub-region: GGGGGGAGCCCGAAACAGCCCTTTGCAGCTCTGCACC |
| SEQ ID NO 563 | Homo sapiens CARD9 - further Δ11 target sub-region: CCTTTGCAGCTCTGCACCAGGAGCAGGTTT |
| SEQ ID NO 564 | Homo sapiens CARD9 - further Δ11 target sub-region: AGGTTTTGCGGAACCCCCATGTA |
| SEQ ID NO 565 | Homo sapiens CARD9 - further Δ11 target sub-region: GGAACCCCCATGTAAGGCTTCCCCGG |

Exemplary exon and Intron Regions of human CARD 9 pre-mRNA - as illustrated with reference to SEQ ID NO 1

| Exon | start_SEQ ID NO 1 | end_SEQ ID NO 1 |
|---|---|---|
| Ex_1 | 1 | 150 |
| Ex 2 | 1588 | 1787 |
| Ex_3 | 2221 | 2358 |
| Ex_4 | 2537 | 2841 |
| Ex_5 | 2981 | 3160 |
| Ex_6 | 3245 | 3388 |
| Ex_7 | 3807 | 3932 |
| Ex_8 | 5854 | 6045 |
| Ex_9 | 6425 | 6466 |
| Ex_10 | 6837 | 6882 |
| Ex_11 | 8465 | 8541 |
| Ex_12 | 9123 | 9199 |
| Ex_13 | 9281 | 9726 |

| | | |
|---|---|---|
| Intron | start_SEQ ID NO 1 | end_SEQ ID NO 1 |
| Int_1 | 151 | 1587 |
| Int_2 | 1788 | 2220 |
| Int_3 | 2359 | 2536 |
| Int_4 | 2842 | 2980 |
| Int_5 | 3161 | 3244 |
| Int_6 | 3389 | 3806 |
| Int_7 | 3933 | 5853 |
| Int_8 | 6046 | 6424 |
| Int_9 | 6467 | 6836 |
| Int_10 | 6883 | 8464 |
| Int_11 | 8542 | 9122 |
| Int_12 | 9200 | 9280 |

A CARD9Δ11 mRNA therefore lacks the Exon 11 region, such as exemplified by nucleotides 8465 - 8541 of SEQ ID No 1, or at least a portion thereof. In some embodiments, the entire exon 11 is lacking in the CARD9Δ11 mRNA, and therefore in some embodiments the Δ11 CARD9 mRNA may be characterized by having a contiguous sequence formed across exon 10 and exon 12. In some embodiments, the CARD9Δ11 mRNA comprises exons 1 to 10, 12 and 13, but lacks exon 11.

### BRIEF DESCRIPTION OF FIGURES

FIG. 1: Splice modulation of CARD9 exon 11 pre-mRNA using exemplary antisense oligonucleotides. The X axis represents to position of each oligonucleotide hybridization to SEQ ID NO 1 (position). The Y axis represents the response which was calculated as (CARD9Δ11^{T}/CARD9WT^{T})/ (CARD9Δ11^{C}/CARD9WT^{C}), wherein CARD9Δ11^{T} and CARD9WT^{T} are the levels of CARD9Δ11 mRNA and CARD9WT mRNA measured from oligo treated cells, respectively, and CARD9Δ11^{C} and CARD9WT^{C} are the levels of CARD9Δ11 mRNA and CARD9WT mRNA measured from untreated (control) cells, respectively (see Examples). Light grey bars represent potential binding sites for serine and arginine rich splicing factors (SRSF). The potential bindings sites are as follows: CGGCGGG, AGCCCGA, CAGCCCU, CACCAGG, AGCAGGU, CCCAUGU, GGCUGCCU, GUUUUGCG, AACCCCCA, UGCAGC, UGCACC and UGCGGA.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotides of the invention are man-made, and are chemically synthesized, and are typically purified or isolated. The oligonucleotides of the invention may comprise one or more modified nucleosides such as 2′ sugar modified nucleosides. The oligonucleotides of the invention may comprise one or more modified internucleoside linkages, such as one or more phosphorothioate internucleoside linkages.

### Antisense Oligonucleotides

The term “antisense oligonucleotide” as used herein is defined as an oligonucleotide capable of modulating expression of a target gene by hybridizing to a target nucleic acid, in particular to a contiguous sequence on a target nucleic acid. Antisense oligonucleotides are not essentially double stranded and are therefore not siRNAs or shRNAs. The antisense oligonucleotides of the present invention may be single stranded. It is understood that single stranded oligonucleotides of the present invention can form hairpins or intermolecular duplex structures (duplex between two molecules of the same oligonucleotide), as long as the degree of intra or inter self-complementarity is less than approximately 50% across of the full length of the oligonucleotide.

In some embodiments, the single stranded antisense oligonucleotides of the invention may not contain RNA nucleosides.

Advantageously, the antisense oligonucleotides of the invention comprise one or more modified nucleosides or nucleotides, such as 2′ sugar modified nucleosides. Furthermore, in some antisense oligonucleotides of the invention, it may be advantageous that the nucleosides which are not modified are DNA nucleosides.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleosides of the oligonucleotide constitute the contiguous nucleotide sequence. The contiguous nucleotide sequence is the sequence of nucleotides in the oligonucleotide of the invention which are complementary to, and in some instances fully complementary to, the target nucleic acid or target sequence.

In some embodiments the oligonucleotide comprises the contiguous nucleotide sequence, such as a F-G-F′ gapmer region or a splice modulating region, and may optionally comprise further nucleotide(s), for example a nucleotide linker region which may be used to attach a functional group (e.g. a conjugate group) to the contiguous nucleotide sequence. The nucleotide linker region may or may not be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleosides of the antisense oligonucleotides of the invention comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing. Exemplary modified nucleosides which may be used in the compounds of the invention include LNA, 2′-O-MOE and morpholino nucleoside analogues.

### Modified Internucleoside Linkage

The term “modified internucleoside linkage” is defined as generally understood by the skilled person as linkages other than phosphodiester (PO) linkages, that covalently couple two nucleosides together. The oligonucleotides of the invention may therefore comprise one or more modified internucleoside linkages such as one or more phosphorothioate internucleoside linkage.

In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Advantageously, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester and phosphorothioate), for example an alkyl phosphonate/methyl phosphonate internucleoside linkage, which according to EP 2 742 135 may for example be tolerated in an otherwise DNA phosphorothioate the gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but which are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide describes an oligonucleotide comprising one or more sugar-modified nucleosides and/or modified internucleoside linkages. The term “chimeric oligonucleotide” is a term that has been used in the literature to describe oligonucleotides comprising sugar modified nucleosides and DNA nucleosides. In some embodiments, it may be advantageous for the antisense oligonucleotide of the invention to be a chimeric oligonucleotide.

### Complementarity

The term “complementarity” describes the capacity for Watson-Crick base-pairing of nucleosides/nucleotides. Watson-Crick base pairs are guanine (G)-cytosine (C) and adenine (A) -thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pairs) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity = (Matches x 100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=-RTln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1 M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In some embodiments, oligonucleotides of the present invention hybridize to a target nucleic acid with estimated ΔG° values below -10 kcal for oligonucleotides that are 10-30 nucleotides in length. In some embodiments the degree or strength of hybridization is measured by the standard state Gibbs free energy AG°. The oligonucleotides may hybridize to a target nucleic acid with estimated ΔG° values below the range of -10 kcal, such as below -15 kcal, such as below -20 kcal and such as below -25 kcal for oligonucleotides that are 8-30 nucleotides in length. In some embodiments the oligonucleotides hybridize to a target nucleic acid with an estimated ΔG° value of -10 to -60 kcal, such as -12 to -40, such as from -15 to -30 kcal or-16 to -27 kcal such as -18 to -25 kcal.

### The Target

The term “target” as used herein is used to refer to the mammalian caspase recruitment domain family member 9, referred to herein as CARD9, and also known in the art as CANDF2; hCARD9. CARD9, GENE ID No 64170, is encoded on human Chromosome 9: 136363956..136373681 reverse strand (GRCh38.p12, NC_000009.12) exemplified herein as SEQ ID NO 1. An example of human CARD9 protein is provided herein as SEQ ID NO 3. In the context of the present invention a CARD9 protein which comprises the amino acid sequence encoded by CARD9 exon 11 is referred to as a WT CARD9.

### Target Nucleic Acid

According to the present invention, the target nucleic acid is a nucleic acid which encodes mammalian CARD9, e.g. human CARD9, and may for example be a gene, a RNA, a mRNA, and pre-mRNA, a mature mRNA or a cDNA sequence. The target may therefore be referred to as an CARD9 target nucleic acid. For in vitro and in vivo use, a preferred target nucleic acid is the pre-mRNA encoding CARD9, as illustrated by SEQ ID NO 1 or naturally occurring variant thereof.

### Wtcard9

WTCARD9 refers to the wild-type CARD9, defined herein as a CARD9 protein which comprises the amino acid sequence encoded by exon 11 of CARD9. Accordingly, the WTCARD9 protein shall be a protein which is encoded by a CARD9 mRNA which comprises exon 11. In some embodiments, the WTCARD9 protein is encoded by a CARD9 mRNA which comprises Exons 1 to 13 (as provided in the Exon table above).

### Card9δ11

A CARD9Δ11 is a CARD9 protein (a CARD9Δ11 variant) which lacks one or more amino acids encoded by CARD 9 Exon 11, in some cases it lacks all or essentially all of the amino acids encoded by CARD9 exon 11. For illustration, exon 11 of SEQ ID NO 1, encodes the amino acids shown in SEQ ID NO 10. A Δ11 CARD9 is not a WTCARD9. The terms A11CARD9 and CARD9Δ11 are used interchangeably herein. In some embodiments, the CARD9Δ11 protein is encoded by a CARD9 mRNA lacking exon 11.

The change in ratio of different transcript products (e.g. WTCARD9 vs. A11CARD9) may be measured by comparing mRNA levels, or levels of the corresponding protein products. Anti-CARD9 antibodies which may be used for assaying the protein levels of CARD9 and CARD9Δ11 using monoclonal or polyclonal antibodies raised against CARD9 (e.g. available from Abcams: Ab55950 or Ab133560 raised against the C-terminus of CARD9, or the polyclonal antibodies AF5248-SP and NBP1-76679 sold by RD Systems and Novus, respectively). By way of example the CARD9Δ11 shown in SEQ ID NO 5 has a mass that is approximately 3 kDa less than the WT CARD9 (62 kDa, SEQ ID NO 3) and the two isoform using the western blot analysis system, Protein Simple or conventional western blot analysis.

### Target Sequences

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides of the invention.

In some embodiments, the antisense oligonucleotide of the invention, or the contiguous nucleotide sequence thereof, is complementary to a target sequence selected from the group consisting of SEQ ID NO 6, 7 - 9, 11 - 284, or 559 - 565.

In some embodiments, the antisense oligonucleotide of the invention, or the contiguous nucleotide sequence thereof, is complementary to a target sequence having a sequence shown in SEQ ID NO 6.

In some embodiments, the antisense oligonucleotide of the invention, or the contiguous nucleotide sequence thereof, is complementary to a target sequence having a sequence shown in SEQ ID NO 7.

In some embodiments, the antisense oligonucleotide of the invention, or the contiguous nucleotide sequence thereof, is complementary to a target sequence having a sequence shown in SEQ ID NO 8.

In some embodiments, the antisense oligonucleotide of the invention, or the contiguous nucleotide sequence thereof, is complementary to a target sequence having a sequence shown in SEQ ID NO 9.

In some embodiments, the antisense oligonucleotide of the invention, or the contiguous nucleotide sequence thereof, is complementary to a target sequence having a sequence shown in SEQ ID NO 559.

In some embodiments, the antisense oligonucleotide of the invention, or the contiguous nucleotide sequence thereof, is complementary to a target sequence having a sequence shown in SEQ ID NO 560.

In some embodiments, the antisense oligonucleotide of the invention, or the contiguous nucleotide sequence thereof, is complementary to a target sequence having a sequence shown in SEQ ID NO 561.

In some embodiments, the antisense oligonucleotide of the invention, or the contiguous nucleotide sequence thereof, is complementary to a target sequence having a sequence shown in SEQ ID NO 562.

In some embodiments, the antisense oligonucleotide of the invention, or the contiguous nucleotide sequence thereof, is complementary to a target sequence having a sequence shown in SEQ ID NO 563.

In some embodiments, the antisense oligonucleotide of the invention, or the contiguous nucleotide sequence thereof, is complementary to a target sequence having a sequence shown in SEQ ID NO 564.

In some embodiments, the antisense oligonucleotide of the invention, or the contiguous nucleotide sequence thereof, is complementary to a target sequence having a sequence shown in SEQ ID NO 565.

### Target Cell

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell. In some embodiments the target cell is a transgenic animal cell which is expressing the human CARD9 target nucleic acid.

For experimental evaluation a target cell may be used which expresses a nucleic acid which comprises a target sequence. For in vitro evaluation, and for assaying the ability of an oligonucleotide to modulate the splicing of CARD9 pre-mRNA, for example, the target cell may be THP-1 cells.

In some embodiments the target cell is a myeloid cell or a myeloid lineage cell. In some embodiments the target cell is a macrophage. In some embodiments the target cell is an intestinal cell.

Typically, the target cell expresses the CARD9 pre-mRNA , which is processed in the cell to the mature CARD9 mRNA, resulting in the expression of the CARD9 protein (WTCARD9). Advantageously, the compounds of the invention modulate the splicing of the CARD9 pre-mRNA to produce a mature CARD9 mRNA which lacks CARD9 exon 11 (or a portion of exon 11), resulting in the expression of a CARD9Δ11 variant.

In some embodiments the target nucleic acid is SEQ ID NO 1, or a naturally occurring variant thereof.

### Naturally Occurring Variant

The term “naturally occurring variant” refers to variants of CARD9 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95%, or in some embodiments at least 98%, or in some embodiments at least 99% homology to a mammalian CARD9 target nucleic acid, such as SEQ ID NO 1. In some embodiments the naturally occurring variants have at least 99% homology to the human CARD9 target nucleic acid of SEQ ID NO: 1.

### Inhibition of Expression of WTCARD9

In some embodiments, the oligonucleotides of the invention, when present in a target cell, inhibit, i.e. decrease expression of WTCARD9. The term “Inhibition of expression” as used herein is to be understood as an overall term for an oligonucleotide’s ability to inhibit, i.e. to decrease the amount or the activity of WTCARD9 in a target cell. In some embodiments, the decrease in amount or activity is at least 10%, 20%, 30%, 40% or 50% compared to that of control cells. Inhibition of activity may be measured by determined by measuring the level of WTCARD9 mRNA, or by measuring the level or activity of WTCARD9 in a cell. Inhibition of expression may therefore be determined in vitro or in vivo. It will be understood that splice modulation may result in an inhibition of expression of WTCARD9 in the cell.

Typically, inhibition of expression is determined by determining the level of the target nucleic acid present in or the activity of the encoded protein product, following the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermined or otherwise known expression level)..

### Enhancing Expression of CARD9Δ11 / CARD9Δ11 Enhancer

In some embodiments, the compounds of the invention, when present in a target cell, may enhance the expression of a CARD9Δ11 mRNA or CARD9Δ11 protein, via modulation of the splicing of exon 11 in the CARD9 pre-mRNA. Modulation of exon11 splicing may be determined by measuring the amount of CARD9Δ11 mRNA or CARD9Δ11 protein in oligo treated cells and in untreated control cells. Thus, the amount of CARD9Δ11 mRNA or CARD9Δ11 protein in the target cell shall be increased as compared to the amount in a control cell. In some embodiments, the increase of the amount of CARD9Δ11 mRNA or CARD9Δ11 protein is at least 10%, 20%, 30%, 40% or 50% compared to the amount of control cells. In some embodiments, the increase of the amount of CARD9Δ11 mRNA or CARD9Δ11 protein is at least 100% as compared to the amount of control cells. In some embodiments, the increase of the amount of CARD9Δ11 mRNA or CARD9Δ11 protein is at least 150% as compared to the amount of control cells. In some embodiments, the increase of the amount of CARD9Δ11 mRNA or CARD9Δ11 protein is at least 200% as compared to the amount of control cells. In some embodiments, the increase of the amount of CARD9Δ11 mRNA or CARD9Δ11 protein is at least 250% as compared to the amount of control cells.

Accordingly, the oligonucleotides of the present invention are A11CARD9 enhancers, i.e. oligonucleotides which result in an increased expression of Δ11CARD9 mRNA or protein after administration of an effective amount of the oligonucleotide to a target cell.

A Δ11CARD9 enhancer may be identified by measuring the increased expression of Δ11CARD9 mRNA or Δ11CARD9 protein as compared to control cells, or by measuring a increased ratio of expression of Δ11CARD9 as compared to WTCARD9 at the mRNA or protein level. As illustrated in the examples this may be determined by comparing the ratio of expression of Δ11CARD9 / WTCARD9 mRNA in cells treated with Δ11CARD9 enhancers to the ratio of expression of Δ11CARD9 / WTCARD9 mRNA in untreated cells, (untreated control response = 1). The comparison may be carried by calculating a ratio between the ratio in treated cells and the ratio untreated cells. E.g., the ratio may be calculated as follows:

(CARD9Δ11^{T}/CARD9WT^{T})/(CARD9Δ11^{C}/CARD9WT^{C}) wherein
CARD9Δ11^{T} is the level of CARD9Δ11 mRNA measured in oligo treated cells
CARD9WT^{T} is the level of CARD9WT mRNA measured in oligo treated cells
CARD9Δ11^{C} is the level of CARD9Δ11 mRNA measured in untreated cells (control)
CARD9WT^{C} is the level of CARD9WT mRNA measured in untreated cells (control)

A response > 1 is therefore indicative of an effective Δ11CARD9 enhancer. Advantageously, the Δ11CARD9 enhancer is capable of eliciting a response of > about 1.5, or > about 2, or > about 2.5..

For example, the increase of expression of Δ11CARD9 mRNA or protein may be determined in THP-1 cells (e.g. as illustrated in the examples).Thus, the oligo treated cells and the untreated control cells may be cultivated as described in the Examples section in order to assess whether there is an increase in CARD9Δ11 mRNA or CARD9Δ11 protein, or not.

In some embodiments, the compounds of the invention are capable of both i) increasing the amount of CARD9Δ11 mRNA or CARD9Δ11 protein in the target cell and ii) decreasing the amount of WTCARD9 mRNA and WTCARD9 protein in a target cell.

### Control Cell

The choice of suitable control cells is a routine part of an experimental setup. For example, the control cell may be a cell which corresponds to the target cell, but which does not comprise the oligonucleotide of the invention, i.e. a cell which has not been contacted with the oligonucleotide of the invention. For example, the control cell may be an untreated THP-1 cell. It is to be understood that the control cells are cultivated under equal conditions as the target cells that comprise the oligonucleotides as described herein.

### Splice Modulation

Splice modulation can be used to correct cryptic splicing, modulate alternative splicing, restore the open reading frame, and induce protein knockdown. In the context of the present invention, a preferred modulation is to modulate alternative splicing to generate a CARD9Δ11 mRNA and thereby enhance expression of CARD9Δ11 protein.

Splice modulation may be assayed by RNA sequencing (RNA-Seq), which allows for a quantitative assessment of the different splice products of a pre-mRNA. In some embodiments of the invention, the antisense oligonucleotides modulate the splicing of the CARD9 pre-mRNA so as to reduce the level of mature CARD9 mRNA which comprises an exon 11 (WTCARD9 mRNA), and to increase the expression of the level of mature CARD9 mRNA which lacks exon1 1 (Δ11CARD9 mRNA).

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention may result in an increase in melting temperature between +0.5 to +12° C., in some instances between +1.5 to +10° C. and in others between +3 to +8° C. per modified nucleoside. Numerous high affinity modified nucleosides are known in the art and include, for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomers of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example, be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′ - 4′ biradical bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-0-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

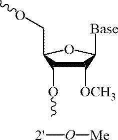

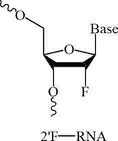

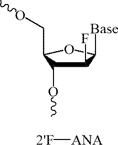

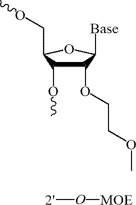

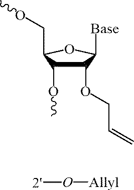

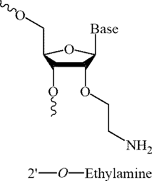

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′ - modified nucleoside which comprises a biradical linking the C2′ and C4′ of the ribose sugar ring of said nucleoside (also referred to as a “2′- 4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76,

Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

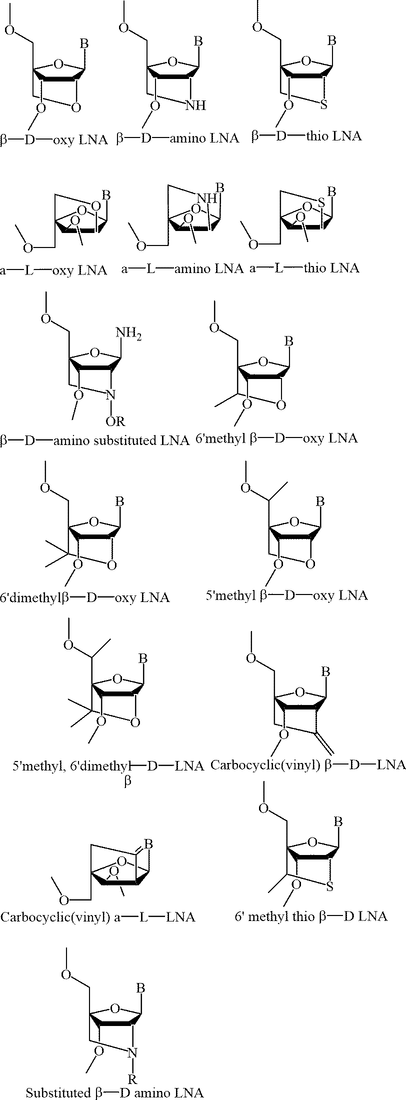

Particular LNA nucleosides are beta-D-oxy-LNA, 6′-methyl-beta-D-oxy LNA such as (S)-6′-methyl-beta-D-oxy-LNA (ScET) and ENA.

In some embodiments, LNA is beta-D-oxy-LNA.

### Morpholino Oligonucleotides

In some embodiments, the oligonucleotide of the invention comprises or consists of morpholino nucleosides (i.e. is a Morpholino oligomer and as a phosphorodiamidate Morpholino oligomer (PMO)). Splice modulating morpholino oligonucleotides have been approved for clinical use -see for example eteplirsen, a 30nt morpholino oligonucleotide targeting a frame shift mutation in DMD, used to treat Duchenne muscular dystrophy. Morpholino oligonucleotides have nucleobases attached to six membered morpholine rings rather ribose, such as methylenemorpholine rings linked through phosphorodiamidate groups, for example as illustrated by the following illustration of 4 consecutive morpholino nucleotides:

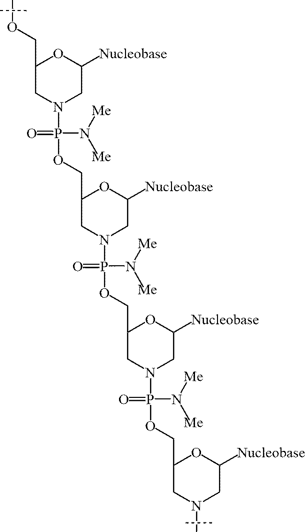

In some embodiments, morpholino oligonucleotides of the invention may be, for example 20 - 40 morpholino nucleotides in length, such as morpholino 25 - 35 nucleotides in length.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, at least 10%, or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91 - 95 of WO01/23613 (hereby incorporated by reference). DNA oligonucleotides are known to effectively recruit RNaseH, as are gapmer oligonucleotides which comprise a region of DNA nucleosides (typically at least 5 or 6 contiguous DNA nucleosides), flanked 5′ and 3′ by regions comprising 2′ sugar modified nucleosides, typically high affinity 2′ sugar modified nucleosides, such as 2-O-MOE and/or LNA. For effective modulation of splicing, degradation of the pre-mRNA is not desirable, and as such it is preferable to avoid the RNaseH degradation of the target. Therefore, the splice modulating oligonucleotides of the invention is preferably not gapmer oligonucleotide. RNaseH recruitment may be avoided by limiting the number of contiguous DNA nucleotides in the oligonucleotide -therefore for effective splice modulation mimxers and totalmers designs may therefore be used.

### Mixmers and Totalmers

For splice modulation it is often advantageous to use antisense oligonucleotides which do not recruit RNAaseH. As RNaseH activity requires a contiguous sequence of DNA nucleotides, RNaseH activity of antisense oligonucleotide may be achieved by designing antisense oligonucleotides which do not comprise a region of more than 3 or more than 4 contiguous DNA nucleosides. This may be achieved by using antisense oligonucleotides or contiguous nucleoside regions thereof with a mixmer design, which comprise sugar modified nucleosides, such as 2′ sugar modified nucleosides, and short regions of DNA nucleosides, such as 1, 2 or 3 DNA nucleosides. Mixmers are exemplified herein by every second design, wherein the nucleosides alternative between 1 LNA and 1 DNA nucleoside, e.g. LDLDLDLDLDLDLDLL, with 5′ and 3′ terminal LNA nucleosides, and every third design, such as LDDLDDLDDLDDLDDL, where every third nucleoside is a LNA nucleoside.

A totalmer is an antisense oligonucleotide or a contiguous nucleotide sequence thereof which does not comprise DNA or RNA nucleosides, and may for example comprise only 2′-O-MOE nucleosides, such as a fully MOE phosphorothioate, e.g. MMMMMMMMMMMMMMMMMMMM, where M = 2′-O-MOE, which are reported to be effective splice modulators for therapeutic use. Alternatively, a mixmer may comprise a mixture of modified nucleosides, such as MLMLMLMLMLMLMLMLMLML, wherein L = LNA and M = 2′-O-MOE nucleosides.

Advantageously, the internucleoside nucleosides in mixmers and totalmers may be phosphorothioate, or a majority of nucleoside linkages in mixmers may be phosphorothioate. Mixmers and totalmers may comprise other internucleoside linkages, such as phosphodiester or phosphorodithioate, by way of example.

### Region D′ or D″ in an Oligonucleotide

The oligonucleotide of the invention may in some embodiments comprise or consist of the contiguous nucleotide sequence of the oligonucleotide which is complementary to the target nucleic acid, such as a mixmer or toalmer region, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the mixmer or totoalmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In one embodiment the oligonucleotide of the invention comprises a region D′ and/or D″ in addition to the contiguous nucleotide sequence which constitutes a mixmer or a totalmer.

In some embodiments the internucleoside linkage positioned between region D′ or D″ and the mixmer or totalmer region is a phosphodiester linkage.

### Conjugate

The term conjugate as used herein refers to an oligonucleotide which is covalently linked to a non-nucleotide moiety (conjugate moiety or region C or third region). The conjugate moiety may be covalentaly linked to the antisense oligonucleotide, optionally via a linker group, such as region D′ or D″.

Oligonucleotide conjugates and their synthesis has also been reported in comprehensive reviews by Manoharan in Antisense Drug Technology, Principles, Strategies, and Applications, S.T. Crooke, ed., Ch. 16, Marcel Dekker, Inc., 2001 and Manoharan, Antisense and Nucleic Acid Drug Development, 2002, 12, 103.

In some embodiments, the non-nucleotide moiety (conjugate moiety) is selected from the group consisting of carbohydrates (e.g. GalNAc), cell surface receptor ligands, drug substances, hormones, lipophilic substances, polymers, proteins, peptides, toxins (e.g. bacterial toxins), vitamins, viral proteins (e.g. capsids) or combinations thereof.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages,. Phosphodiester containing biocleavable linkers are described in more detail in WO 2014/076195.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2 - C36 amino alkyl group, including, for example C6 to C12 amino alkyl groups. In some embodiments the linker (region Y) is a C6 amino alkyl group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

### The Oligonucleotides of the Invention

The invention provides for an antisense oligonucleotide of 10 to 40, such as 10 to 30 nucleotides in length, for modulating the splicing of a mammalian CARD9 pre-mRNA transcript, wherein said oligonucleotide comprises a contiguous nucleotide sequence of at least 10 nucleotides in length with at least 90% complementarity, and in some instances 100% complementarity, to SEQ ID NO 1.

The invention provides for an antisense oligonucleotide of 10 to 40, such as 10 to 30 nucleotides in length, for modulating the splicing of a mammalian CARD9 pre-mRNA transcript, wherein said oligonucleotide comprises a contiguous nucleotide sequence of at least 10 nucleotides in length with at least 90% complementarity, and in some instances 100% complementarity, to SEQ ID NO 2.

In some embodiments, the antisense oligonucleotide or contiguous nucleotide sequence thereof, is complementary to, and in some instances, fully complementary to SEQ ID NO 6.

In some embodiments, the antisense oligonucleotide or contiguous nucleotide sequence thereof, is complementary to, and in some instances, fully complementary to SEQ ID NO 7.

In some embodiments, the antisense oligonucleotide or contiguous nucleotide sequence thereof, is complementary to, and in some instances, fully complementary to SEQ ID NO 8.

In some embodiments, the antisense oligonucleotide or contiguous nucleotide sequence thereof, is complementary to, and in some instances, fully complementary to SEQ ID NO 9.

In some embodiments, the antisense oligonucleotide or contiguous nucleotide sequence thereof, is complementary to, and in some instances, fully complementary to SEQ ID NO 559.

In some embodiments, the antisense oligonucleotide or contiguous nucleotide sequence thereof, is complementary to, and in some instances, fully complementary to SEQ ID NO 560.

In some embodiments, the antisense oligonucleotide or contiguous nucleotide sequence thereof, is complementary to, and in some instances, fully complementary to SEQ ID NO 561.

In some embodiments, the antisense oligonucleotide or contiguous nucleotide sequence thereof, is complementary to, and in some instances, fully complementary to SEQ ID NO 562.

In some embodiments, the antisense oligonucleotide or contiguous nucleotide sequence thereof, is complementary to, and in some instances, fully complementary to SEQ ID NO 563.

In some embodiments, the antisense oligonucleotide or contiguous nucleotide sequence thereof, is complementary to, and in some instances, fully complementary to SEQ ID NO 564.

In some embodiments, the antisense oligonucleotide or contiguous nucleotide sequence thereof, is complementary to, and in some instances, fully complementary to SEQ ID NO 565.

In some embodiments, the antisense oligonucleotide or contiguous nucleotide sequence thereof, is complementary to a sequence selected from the group consisting of SEQ ID NO 11 - 284.

In some embodiments, the antisense oligonucleotide is capable of enhancing the expression of CARD9Δ11.

In some embodiments, the antisense oligonucleotide is capable of reducing the expression of WTCARD9.

In some embodiments, the antisense oligonucleotide is or comprises an antisense oligonucleotide mixmer or totalmer.

In some embodiments, the antisense oligonucleotide is a morpholino oligonucleotide.

In some embodiments, the antisense oligonucleotide is a 2′-O-MOE oligonucleotide, i.e. comprises one or more 2′-O-MOE nucleosides.

In some embodiments, the antisense oligonucleotide is an LNA oligonucleotide, i.e. comprises one or more LNA nucleosides.

In some embodiments, the antisense oligonucleotide or contiguous nucleotide sequence thereof is 10 - 25 or 10 - 20 nucleotides in length.

In some embodiments, the contiguous nucleotide sequence of the antisense oligonucleotide of the present invention comprises one or more nucleotide sequences which is complementary to, such as fully complementary to a sequence selected from the group consisting of CGGCGGG, AGCCCGA, CAGCCCU, CACCAGG, AGCAGGU, CCCAUGU, GGCUGCCU, GUUUUGCG, AACCCCCA, UGCAGC, UGCACC and UGCGGA. It will be understood that these 6 - 9nt sequences are present within an advantageous region (target site sequence) of SEQ ID NO 6 (nucleotide 200 - 280 of SEQ ID NO 6) and that several of these sequence overlap within this target site sequence. In some embodiments, the antisense oligonucleotide of the invention may therefore comprise a contiguous nucleotide sequence of 10 - 40 nucleotides in length which is complementary to, such as fully complementary to nucleotides 200 - 280 of SEQ ID NO 6. In some embodiments, the antisense oligonucleotide of the invention may therefore comprise a contiguous nucleotide sequence of 12 - 25 nucleotides in length which is complementary to, such as fully complementary to nucleotides 200 - 280 of SEQ ID NO 6. It will be recognised that a region of the contiguous nucleotide sequence may comprise the complement of more than one of the sequences selected from the group consisting of CGGCGGG, AGCCCGA, CAGCCCU, CACCAGG, AGCAGGU, CCCAUGU, GGCUGCCU, GUUUUGCG, AACCCCCA, UGCAGC, UGCACC and UGCGGA. In some embodiments, the oligonucleotides of the invention may comprise the full complement of at least 2 or at least 3 of the sequences selected from the group consisting of CGGCGGG, AGCCCGA, CAGCCCU, CACCAGG, AGCAGGU, CCCAUGU, GGCUGCCU, GUUUUGCG, AACCCCCA, UGCAGC, UGCACC and UGCGGA.

In some embodiments, the contiguous nucleotide sequence of the antisense oligonucleotide consists or comprises of a sequence selected from SEQ ID NO 285-558.

In some embodiments, the antisense oligonucleotide consists or comprises of an oligonucleotide provided herein, such as a compound selected from the group consisting of compound ID NO #285_1 - #558_1 (see table in the Examples section).

In some advantageous embodiments the antisense oligonucleotide of the invention is in the form of a pharmaceutically acceptable salt, such as a sodium salt or a potassium salt.

The invention further provides for a pharmaceutically acceptable salt of the antisense oligonucleotide or conjugate according to the invention.

The invention further provides for a conjugate comprising the antisense oligonucleotide according to the invention, and at least one conjugate moiety covalently attached to said oligonucleotide.

The invention further provides for a pharmaceutical composition comprising the antisense oligonucleotide or the conjugate of the invention and a pharmaceutically acceptable diluent, solvent, carrier, salt and/or adjuvant.

The invention provides for a method for modulating the splicing of a CARD9 pre-mRNA in a cell which is expressing CARD9 (e.g. a target cell), said method comprising administering an antisense oligonucleotide or the conjugate or the pharmaceutical composition, of the invention in an effective amount to said cell. The method may be in vitro method or an in vivo method.

In some embodiments of the method of the invention, the administration of the antisense oligonucleotide results in enhanced expression of a CARD9Δ11 variant.

In some embodiments of the method of the invention, the administration of the antisense oligonucleotide results in reduced expression of WTCARD9.

In some embodiments, the administration of the antisense oligonucleotide results in reduced expression of WTCARD9 and enhanced expression of a CARD9Δ11 variant.

In some embodiments, the cell, such as the target cell is a mammalian cell. In some embodiments, the cell is a human cell.

In some embodiments the CARD9 target is a human CARD9. In some embodiments the CARD9 target nucleic acid is a human CARD9 pre-mRNA such as that illustrated as SEQ ID NO 1.

The invention provides for a method for treating or preventing an inflammatory disease comprising administering a therapeutically or prophylactically effective amount of the antisense oligonucleotide or the conjugate or the pharmaceutical composition, of the invention to a subject suffering from or susceptible to the inflammatory disease, such as inflammatory bowel disease.

The invention provides for an oligonucleotide or the conjugate or pharmaceutical composition of the invention for use as a medicament.

The invention provides for an antisense oligonucleotide or the conjugate or pharmaceutical composition of the invention for use in the treatment of an inflammatory disease, such as inflammatory bowel disease.

The invention provides for the use of an antisense oligonucleotide or the conjugate or pharmaceutical composition of the invention for use the preparation of a medicament for treatment or prevention of an inflammatory disease, such as inflammatory bowel disease.

In some embodiments, the oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the oligonucleotide of the invention, or contiguous nucleotide sequence thereof is fully complementary (100% complementary) to a region of the target nucleic acid, or in some embodiments may comprise one or two mismatches between the oligonucleotide and the target nucleic acid.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The oligonucleotides of the invention are designed with modified nucleosides and DNA nucleosides. In some examples, high affinity modified nucleosides are used.

Suitable modifications are described in the “Definitions” section under “modified nucleoside”, “high affinity modified nucleosides”, “sugar modifications”, “2′ sugar modifications” and Locked nucleic acids (LNA)″.

In an embodiment, the oligonucleotide comprises one or more sugar modified nucleosides, such as 2′ sugar modified nucleosides. Preferably the oligonucleotides of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. In some embodiments, one or more of the modified nucleoside(s) may be a locked nucleic acid (LNA).

In a further embodiment the oligonucleotide comprises at least one modified internucleoside linkage. Suitable internucleoside modifications are described in the “Definitions” section under “Modified internucleoside linkage”. In some oligonucleotides of the invention, at least 75% of the internucleoside linkages within the contiguous nucleotide sequence are phosphorothioate internucleoside linkages. In some embodiments all of the internucleotide linkages in the contiguous nucleotide sequence of the oligonucleotide are phosphorothioate linkages.

In some embodiments the oligonucleotides of the invention are capable of inhibiting the expression of the WTCARD9 target in a cell which is expressing the CARD9 target (a target cell), for example either in vivo or in vitro.

The contiguous sequence of nucleobases of the oligonucleotide of the invention is typically complementary to the CARD9 target nucleic acid, such as SEQ ID NO 1, or target sequence (such as a sequence selected from the group consisting of SEQ ID NO 6, 7, 8, 9, 559, 560, 561, 562, 563, 564 or 565) or target site region thereof (such as a sequence selected from the group consisting of SEQ ID Nos 10 - 285), as measured across the length of the oligonucleotide, optionally with the exception of a mismatch, and optionally excluding nucleotide based linker regions which may link the oligonucleotide to an optional functional group such as a conjugate, or other non-complementary terminal nucleotides (e.g. region D′ or D″).

Advantageously, the contiguous sequence of nucleobases of the oligonucleotide of the invention comprises a sequence motif as shown in a sequence selected from SEQ ID NO 285 - 558, or at least 10 contiguous nucleotides thereof.

Advantageously, the contiguous sequence of nucleobases of the oligonucleotide of the invention comprises a sequence motif as shown in a sequence selected from SEQ ID NO 285 - 558, or at least 12 contiguous nucleotides thereof.

Advantageously, the contiguous sequence of nucleobases of the oligonucleotide of the invention comprises a sequence motif as shown in a sequence selected from SEQ ID NO 285 - 558, or at least 14 contiguous nucleotides thereof.

In some embodiments, the contiguous sequence of nucleobases of the oligonucleotide of the invention comprises a sequence motif as shown in a sequence selected from SEQ ID NOs 290, 294, 295, 297, 300, 301, 306, 307, 308, 312, 317, 318, 319, 322, 329, 333, 337, 341, 352, 353, 358, 373, 374, 378, 379, 380, 387, 393, 394, 396, 399, 400, 405, 406, 407, 408, 409, 411, 413, 413, 414, 414, 414, 415, 415, 415, 416, 417, 417, 417, 418, 422, 424, 425, 426, 429, 430, 431, 433, 434, 435, 436, 439, 440, 441, 442, 443, 446, 447, 448, 449, 450, 452, 453, 454, 455, 456, 458, 459, 460, 461, 462, 464, 471, 472, 490, 519, 530, 533, 541, 542, 546, and 548, or at least 12 contiguous nucleotides thereof.

In some embodiments, the contiguous sequence of nucleobases of the oligonucleotide of the invention comprises a sequence motif as shown in a sequence selected from SEQ ID NOs 319, 322, 396, 405, 408, 413, 414, 414, 429, 431, 440, 442, 446, 448, 449, 450, 452, 453, 454, 455, 456, 459, 460, and 461 or at least 12 contiguous nucleotides thereof.

In some embodiments, the contiguous sequence of nucleobases of the oligonucleotide of the invention comprises a sequence motif as shown in a sequence selected from SEQ ID NOs 413, 414, 448, 449, 450, 454, 456, 459, and 460 or at least 12 contiguous nucleotides thereof.

In some embodiments, the oligonucleotide of the invention is selected from the group consisting of 290_1, 294_1, 295_1, 297_1, 300_1, 301_1, 306_1, 307_1, 308_1, 312 1, 317_1, 318 1, 319 1, 322 1, 329 1, 333 1, 337 1, 341 1, 352 1, 353 1, 358 1, 373 1, 374 1, 378 1, 379 1, 380 1, 387 1, 393 1, 394 1, 396 1, 399 1, 400 1, 405 1, 406 1, 407 1, 408 1, 409 1, 411 1, 413_1, 413_2, 414_1, 414_2, 414_3, 4151, 415_2, 415 3, 4161, 4171, 417_2, 417_3, 4181, 422 1, 424 1, 425 1, 426 1, 429 1, 430 1, 431 1, 433 1, 434 1, 435 1, 436 1, 439 1, 440 1, 441 1, 442 1, 443 1, 446 1, 447 1, 448 1, 449 1, 450 1, 452 1, 453 1, 454 1, 455 1, 456 1, 458 1, 459 1, 460 1, 461 1, 462 1, 464 1, 471 1, 472 1, 490 1, 519 1, 530 1, 533 1, 541 1, 542_1, 546_1, and 548 1.

In some embodiments, the oligonucleotide of the invention is selected from the group consisting of 319_1, 322_1, 396_1, 405_1, 408_1, 413_2, 414_2, 414_3, 429_1, 431_1, 440_1, 442_1, 446 1, 448 1, 449 1, 450 1, 452 1, 453 1, 454 1, 455 1, 456 1, 459 1, 460 1, and 461_1.

In some embodiments, the oligonucleotide of the invention is selected from the group consisting of 413 2, 414 2, 448_1, 449_1, 450_1, 454_1, 456_1, 459_1, and 460_1.

### Pharmaceutically Acceptable Salts

In a further aspect the invention provides a pharmaceutically acceptable salt of the antisense oligonucleotide or a conjugate thereof, such as a pharmaceutically acceptable sodium salt or potassium salt.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50 - 300 µM solution.

### Applications

The oligonucleotides of the invention may be utilized as research reagents for, for example, diagnostics, therapeutics and prophylaxis.

In research, such oligonucleotides may be used to specifically modulate the synthesis of CARD9 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the oligonucleotide of the invention in research or diagnostics the target nucleic acid may be a cDNA or a synthetic nucleic acid derived from DNA or RNA.

The present invention provides an in vivo or in vitro method for modulating CARD9 expression, such as splice modulation, in a target cell which is expressing CARD9, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal. In some embodiments the target cell is a myeloid cell or a myeloid lineage cell. In some embodiments the target cell is a macrophage. In some embodiments the target cell is present in, isolated from, or derived from an intestinal tissue.

In diagnostics the oligonucleotides may be used to detect and quantitate CARD9 expression in cell and tissues by northern blotting, in-situ hybridisation or similar techniques.

For therapeutics, the oligonucleotides may be administered to an animal or a human, suspected of having a disease or disorder, which can be treated by modulating the expression of CARD9. Such diseases or disorders include inflammatory bowel disease (such as Crohn’s disease and ulcerative colitis), pancreatitis, IgA nephropathy, primary sclerosing cholangitis, cardiovascular disease, cancer and diabetes.

The invention provides methods for treating or preventing a disease or disorder, comprising administering a therapeutically or prophylactically effective amount of an oligonucleotide, an oligonucleotide conjugate or a pharmaceutical composition of the invention to a subject suffering from or susceptible to the disease or disorder, such as diseases or disorders selected from the group consisting of inflammatory bowel disease (such as Crohn’s disease and ulcerative colitis), pancreatitis, IgA nephropathy, primary sclerosing cholangitis, cardiovascular disease, cancer and diabetes.

The methods of the invention are preferably employed for treatment or prophylaxis against diseases caused by abnormal levels and/or activity of CARD9.

In some embodiments the disease is an inflammatory disease.

In some embodiments, the disease is Inflammatory bowel disease. For example, the inflammatory bowel disease is Crohn’s disease. Alternatively, the inflammatory bowel disease is ulcerative colitis.

### Administration

In some embodiments, the oligonucleotides or pharmaceutical compositions of the present invention may, for example be administered by a parenteral route including intravenous, intraarterial, subcutaneous, intraperitoneal or intramuscular injection or infusion. In some embodiments the oligonucleotide or oligonucleotide conjugate is administered intravenously. In another embodiment the oligonucleotide or oligonucleotide conjugate is administered subcutaneously.

In some embodiments, the oligonucleotides or pharmaceutical compositions of the present invention are administered orally or rectally. In some embodiments, the oligonucleotides or pharmaceutical compositions of the present invention are administered orally or rectally for the treatment of inflammatory bowel disease, such as Crohn’s disease or ulcerative colitis

### Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

### EXAMPLES

### Example 1

Oligonucleotides #285_1 - #558_1 were synthesized and their ability to modulate the splicing of the CARD9 pre-mRNA to enhance the ratio of Δ11CARD9 mRNA as compared to WTCARD9 mRNA was evaluated. Specifically, it was evaluated whether the oligonucleotides enhance the level of Δ11CARD9 mRNA as compared to the level of Δ11CARD9 mRNA in untreated cells.

### Materials and Methods

Cells: 50.000 THP-1 cells were seeded in 96 well plates and oligos (Table 1) added to obtain a gymnotic uptake. Final oligo concentration in the media was 25 µM. Cells were incubated for 7 days at at 37° C. and 5% CO2. The target sequence that were covered by mixmers that is tiled be oligos targeting SEQ ID NO 6. As control, untreated THP-1 cells were used.

Droplet digital PCR was performed BioRads QX200 Droplet Digital PCR (ddPCRTM) system using the following primers and probes:
Loading control for RNA input:
Hs.PT.58v.45621572 - Predesigned HEX based assay targeting exon 8-9 of HTRP1.

For the CARD9Δ11 transcript:

Hs. CARD9Δ11 probe /56-
FAM/CTCAGACAA/ZEN/AGGACGCAGGCCTG/3IABkFQ/
Primer 1 AGTTCTCAAAACTCTCTTTGAGGC
Primer 2 GGAAGATGGCTCACCCAG

The response was calculated by the following formula:

(CARD9Δ11^{T}/CARD9WT^{T})/(CARD9A11^{C}/CARD9WT^{C}) wherein
CARD9Δ11^{T} is the level of CARD9Δ11 mRNA measured in oligo treated cells
CARD9WT^{T} is the level of CARD9WT mRNA measured in oligo treated cells
CARD9Δ11^{C} is the level of CARD9Δ11 mRNA measured in untreated cells (control)
CARD9WT^{C} is the level of CARD9WT mRNA measured in untreated cells (control)

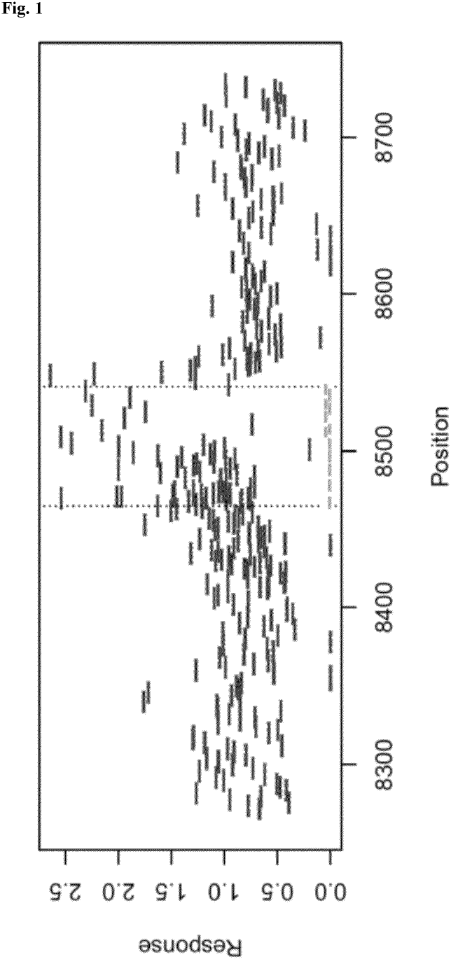

The response is shown in FIG. 1 (see dark grey bars). In principle, the response reflects the increase in the proportion of CARD9Δ11 mRNA as compared to untreated controls. Accordingly, a response of 2 indicates that twice as much CARD9d11 mRNA is present in treated cells as compared to untreated control cells, a response of 3 is 3 times the level in untreated control. In other words, the response refers to the increase in CARD9Δ11 skipping of oligo treated cells, compared to the untreated cells (in THP-1 cells there is some CARD9Δ11 events seen without oligo treatment).

### Compound and Data Table

The motif of nucleobases in the oligonucleotides is shown as SEQ ID NO 285 - 558. The compounds are designated as a compound ID number (#), with the oligonucleotide structure (5′ -3′), wherein capital letters designate a beta-D-oxy LNA nucleoside, a lower case letter designates a DNA nucleoside, capital C designates 5-methyl cytosine beta-D-oxy LNA nucleoside, and a superscript m before a lower case c represent a 5-methyl cytosine DNA nucleoside (see e.g. SEQ ID NO 294), and all internucleoside linkages are phosphorothioate internucleoside linkages.

| SEQ_ID_NO | CMP_ID_NO (#) | Oligo_structure | Response |
|---|---|---|---|
| 285 | 285_1 | TcaTTgCTcCAgGA | 0.67 |
| 286 | 286_1 | GTtCAtTgcTCcAG | 0.78 |
| 287 | 287_1 | CTgTtcATtGCtCC | 0.39 |
| 288 | 288_1 | GCctGTtCatTgCT | 0.95 |
| 289 | 289_1 | TtgCCtGTtCAtTG | 0.66 |
| 290 | 290_1 | GCttGCctGTtcAT | 1.27 |
| 291 | 291_1 | TCgCTtgCcTgtTC | 0.41 |
| 292 | 292_1 | CCtcGcTtgCCtGT | 0.47 |
| 293 | 293_1 | GgCCtcGcTTgcCT | 0.51 |
| 294 | 294_1 | ATgGcCT^{m}cgCTtGC | 1.01 |
| 295 | 295_1 | TTaTgGCctCGcTT | 1.08 |
| 296 | 296_1 | CCttATggCCtcGC | 0.62 |
| 297 | 297_1 | GgCCttATgGccTC | 1.24 |
| 298 | 298_1 | GAgGcCTtATggCC | 0.73 |
| 299 | 299_1 | CTgAggCCtTatGG | 0.92 |
| 300 | 300_1 | GaCTgAGgCCttAT | 1.06 |
| 301 | 301_1 | GAgaCTgAGgCcTT | 1.17 |
| 302 | 302_1 | ATgAgaCTgAGgCC | 0.8 |
| 303 | 303_1 | AgaTGaGAcTGaGG | 0.91 |
| 304 | 304_1 | GTaGAtGAgACtGA | 0.97 |
| 305 | 305_1 | CTgTAgATgAGaCT | 0.46 |
| 306 | 306_1 | TTcTGtAGaTGaGA | 1.18 |
| 307 | 307_1 | CTtTCtGTaGAtGA | 1.3 |
| 308 | 308_1 | TGcTTtCTgTAgAT | 1.29 |
| 309 | 309_1 | TCtgCTtTcTGtAG | 0.58 |
| 310 | 310_1 | ACtCTgCTttCtGT | 0.49 |
| 311 | 311_1 | AGaCtCTgCTttCT | 0.7 |
| 312 | 312_1 | GTaGAcTctGCtTT | 1.06 |
| 313 | 313_1 | GTgTaGActCTgCT | 0.85 |
| 314 | 314_1 | CaGTgTAgaCTcTG | 0.71 |
| 315 | 315_1 | TcCaGTgTaGAcTC | 0.95 |
| 316 | 316_1 | GgtCCaGTgTagAC | 0.47 |
| 317 | 317_1 | TgGgtCCaGTgtAG | 1.06 |
| 318 | 318_1 | CAtgGgtCCaGtGT | 1.07 |
| 319 | 319_1 | CacATgGGtCCaGT | 1.76 |
| 320 | 320_1 | CCcAcATggGTcCA | 0.85 |
| 321 | 321_1 | TccCCacATgGgTC | 0.93 |
| 322 | 322_1 | GAtCCccAcATgGG | 1.72 |
| 323 | 323_1 | GggATcCCcACaTG | 0.87 |
| 324 | 324_1 | GTgGgATccCCaCA | 0.89 |
| 325 | 325_1 | TgGTggGAtCccCA | 0.84 |
| 326 | 326_1 | CcTgGTgGgATcCC | 0 |
| 327 | 327_1 | GgcCTgGTgGgaTC | 0 |
| 328 | 328_1 | AaGgCCtgGTggGA | 0.53 |
| 329 | 329_1 | CCaaGgcCTgGtGG | 1.27 |
| 330 | 330_1 | TtcCAaGgCCtgGT | 0.99 |
| 331 | 331_1 | TcTTcCAaGGccTG | 0.72 |
| 332 | 332_1 | TgtCTtCCaaGgCC | 0.58 |
| 333 | 333_1 | GCtGTcTTccAaGG | 1.04 |
| 334 | 334_1 | GGgCTgtCTtCcAA | 0.81 |
| 335 | 335_1 | TgGggCTgtCTtCC | 0.54 |
| 336 | 336_1 | AgTgGggCTgTcTT | 0.6 |
| 337 | 337_1 | GcaGtgGggCTgTC | 1.01 |
| 338 | 338_1 | TtGcaGTgGGgcTG | 0 |
| 339 | 339_1 | CAttGcaGTgGgGC | 0.8 |
| 340 | 340_1 | GGcATtgCaGTgGG | 0.5 |
| 341 | 341_1 | CCgGcATtgCAgTG | 1.01 |
| 342 | 342_1 | CCccGgCatTGcAG | 0.34 |
| 343 | 343_1 | CtCCccGgcATtGC | 0.63 |
| 344 | 344_1 | CccTccCCggCaTT | 0.86 |
| 345 | 345_1 | AcCccTcCccGgCA | 0.56 |
| 346 | 346_1 | CcAcCccTcCccGG | 0.78 |
| 347 | 347_1 | CccCacCccTccCC | 0.35 |
| 348 | 348_1 | TgcCccAccCctCC | 0.41 |
| 349 | 349_1 | GcTgcCccACccCT | 0.41 |
| 350 | 350_1 | CgGcTgcCccAcCC | 0.91 |
| 351 | 351_1 | CAcGgCTgCCccAC | 0.78 |
| 352 | 352_1 | AGcAcGgcTGccCC | 1.09 |
| 353 | 353_1 | ATaGCacGgCTgCC | 1.06 |
| 354 | 354_1 | GTaTaGCacGGcTG | 0.97 |
| 355 | 355_1 | AcGTaTaGCaCgGC | 0.59 |
| 356 | 356_1 | CAcGTaTAgCAcGG | 0.66 |
| 357 | 357_1 | CCaCGtATaGCaCG | 0.58 |
| 358 | 358_1 | GCcACgTAtAGcAC | 1.16 |
| 359 | 359_1 | GgcCAcGTaTAgCA | 0.43 |
| 360 | 360_1 | GggCCacGTaTaGC | 0.42 |
| 361 | 361_1 | TGggCCaCGtAtAG | 0.97 |
| 362 | 362_1 | CTgGGcCAcGTaTA | 0.78 |
| 363 | 363_1 | GcTgGgCCaCGtAT | 0.78 |
| 364 | 364_1 | TgCTgGgcCAmcgTA | 0.59 |
| 365 | 365_1 | CTgCTgGGcCacGT | 0.47 |
| 366 | 366_1 | TcTgCTgGgCcaCG | 0.42 |
| 367 | 367_1 | ATcTgCTggGCcAC | 0.78 |
| 368 | 368_1 | CatCTgCTgGgcCA | 0.81 |
| 369 | 369_1 | CcATcTgCTgGgCC | 0.72 |
| 370 | 370_1 | CccATcTgCTggGC | 0.56 |
| 371 | 371_1 | GccCatCTgCTgGG | 0.93 |
| 372 | 372_1 | TgCCcATctGctGG | 0.66 |
| 373 | 373_1 | CTgCCcATcTgcTG | 1.08 |
| 374 | 374_1 | CCtgCccATcTgCT | 1.03 |
| 375 | 375_1 | AccTgCCcATctGC | 0.6 |
| 376 | 376_1 | GAccTgCCcATcTG | 0.96 |
| 377 | 377_1 | GgAcCTgcCCatCT | 0.76 |
| 378 | 378_1 | GggAccTgCCcaTC | 1.32 |
| 379 | 379_1 | TggGAcCTgCCcAT | 1.06 |
| 380 | 380_1 | GTggGAccTGccCA | 1.12 |
| 381 | 381_1 | TgTggGAccTgcCC | 0.91 |
| 382 | 382_1 | CTgTggGAccTgCC | 0.62 |
| 383 | 383_1 | CctGTgGgACctGC | 0 |
| 384 | 384_1 | GcCTgTggGAccTG | 0.43 |
| 385 | 385_1 | TgcCTgTggGAcCT | 0.68 |
| 386 | 386_1 | GTgcCTgTgGgaCC | 0.87 |
| 387 | 387_1 | CGtgCCtGTgGgAC | 1.23 |
| 388 | 388_1 | TcGTgCCtGTggGA | 0.65 |
| 389 | 389_1 | GTcGTgcCTgTgGG | 0.62 |
| 390 | 390_1 | AGtcGTgcCTgtGG | 0.76 |
| 391 | 391_1 | GaGTcGTgcCTgTG | 0.77 |
| 392 | 392_1 | AgaGTcGTgCCtGT | 0.57 |
| 393 | 393_1 | GAgaGTcGTgCcTG | 1.09 |
| 394 | 394_1 | GgAGaGTcGTgcCT | 1.06 |
| 395 | 395_1 | AGgaGaGTcGTgCC | 0.68 |
| 396 | 396_1 | AagGAgAGtCGtGC | 1.75 |
| 397 | 397_1 | AAaGGaGAgTCgTG | 0.91 |
| 398 | 398_1 | GAaAGgAGaGTcGT | 0.96 |
| 399 | 399_1 | GGaAAgGAgAGtCG | 1.11 |
| 400 | 400_1 | TGgAAaGGaGAgTC | 1.14 |
| 401 | 401_1 | CTgGAaAGgAGaGT | 0.87 |
| 402 | 402_1 | CCtGGaAAgGAgAG | 0.9 |
| 403 | 403_1 | GCcTgGAaaGGaGA | 0.83 |
| 404 | 404_1 | AGcCTgGAaAGgAG | 0.74 |
| 405 | 405_1 | CAgCCtgGAaAgGA | 1.51 |
| 406 | 406_1 | GCaGcCTgGAaaGG | 1.45 |
| 407 | 407_1 | GGcAGcCTgGAaAG | 1.21 |
| 408 | 408_1 | AGgCAgCCtgGaAA | 1.63 |
| 409 | 409_1 | AagGCaGCcTGgAA | 1.27 |
| 410 | 410_1 | CAaGgCAgcCTgGA | 0.85 |
| 411 | 411_1 | GcAAggCagCCtGG | 1.34 |
| 412 | 412_1 | GgCAagGcaGccTG | 0.83 |
| 412 | 412_2 | GgCAaGgcAGccTG | 0.75 |
| 412 | 412_3 | GgCAaGGcaGccTG | 0.98 |
| 413 | 413_1 | CGgcAaGgcAGcCT | 1.17 |
| 413 | 413_2 | CGgcAaGgCAgcCT | 2.54 |
| 413 | 413_3 | CGgCAaGgcAGcCT | 0.78 |
| 414 | 414_1 | CcGgcAaGgCagCC | 1.47 |
| 414 | 414_2 | CcGgcAagGCagCC | 2.02 |
| 414 | 414_3 | CCggCAaGgCagCC | 1.97 |
| 415 | 415_1 | GccGgCAagGcaGC | 1.29 |
| 415 | 415_2 | GccGgCaaGGcaGC | 1.49 |
| 415 | 415_3 | GccGgCAaGGcaGC | 1.22 |
| 416 | 416_1 | CGccGGcaAGgcAG | 1.1 |
| 416 | 416_2 | CGccGgCAaGGcAG | 0.95 |
| 416 | 416_3 | CgCCgGCaaGgcAG | 0.95 |
| 417 | 417_1 | CcGccGgCAaGgCA | 1.47 |
| 417 | 417_2 | CCgCcGgcAaGgCA | 1.05 |
| 417 | 417_3 | CcGcCGgCAaGgCA | 1.01 |
| 418 | 418_1 | CccGCcGgcAAgGC | 1.29 |
| 419 | 419_1 | CccCgCcGgCAaGG | 0.75 |
| 420 | 420_1 | CccCmcgCCggCaAG | 0.72 |
| 421 | 421_1 | CcCcCcGcCGgcAA | 0.71 |
| 422 | 422_1 | TcCcCccGccGgCA | 1.04 |
| 423 | 423_1 | CtcCccCmcgCmcgGC | 0.98 |
| 424 | 424_1 | GctCccCccGccGG | 1.04 |
| 425 | 425_1 | GgcTccCccCgcCG | 1.03 |
| 426 | 426_1 | GggCtcCccCmcgCC | 1.37 |
| 427 | 427_1 | CggGctCccCccGC | 0.72 |
| 428 | 428_1 | TcGggCTccCccCG | 0.88 |
| 429 | 429_1 | TtcGggCTcCccCC | 1.6 |
| 430 | 430_1 | TTtcGggCtCccCC | 1.23 |
| 431 | 431_1 | GTttCgGgcTccCC | 2 |
| 432 | 432_1 | TgTTtcGGgcTcCC | 0.95 |
| 433 | 433_1 | CTgTttCGgGctCC | 1.44 |
| 434 | 434_1 | GctGTttCgGGcTC | 1.29 |
| 435 | 435_1 | GgCTgTTtcGGgCT | 1.26 |
| 436 | 436_1 | GggCTgTTtcGgGC | 1.1 |
| 437 | 437_1 | AGggCTgtTTmcgGG | 0.99 |
| 438 | 438_1 | AAgGGcTGtTTcGG | 0.9 |
| 439 | 439_1 | AaAGgGCtGTttCG | 1.4 |
| 440 | 440_1 | CAaAGgGCtGTtTC | 1.63 |
| 441 | 441_1 | GCaaAgGGcTGtTT | 1.13 |
| 442 | 442_1 | TgCAaAGgGCtgTT | 1.86 |
| 443 | 443_1 | CTgCaaAGgGCtGT | 1.09 |
| 444 | 444_1 | GCtgCAaaGGgcTG | 0.19 |
| 445 | 445_1 | AgCTgCAaaGGgCT | 1 |
| 446 | 446_1 | GAgCTgCAaaGgGC | 2 |
| 447 | 447_1 | AgaGCtGCaaAgGG | 1.2 |
| 448 | 448_1 | CAgaGCtGCaAaGG | 2.45 |
| 449 | 449_1 | GgtGcaGAgCTgCA | 2.54 |
| 450 | 450_1 | TccTgGtgCAgaGC | 2.16 |
| 451 | 451_1 | CTgCTccTgGTgCA | 0.74 |
| 452 | 452_1 | AAaCCtGctCCtGG | 1.94 |
| 453 | 453_1 | CgCAaAaCCtGcTC | 1.74 |
| 454 | 454_1 | GTtCCgCAaAAcCT | 2.25 |
| 455 | 455_1 | TgGGgGTtCCgcAA | 1.89 |
| 456 | 456_1 | TacATgGGgGTtCC | 2.31 |
| 457 | 457_1 | GCcTTacATgGgGG | 0.96 |
| 458 | 458_1 | GGaaGCcTTaCaTG | 1.28 |
| 459 | 459_1 | GGgGAaGcCTtaCA | 2.64 |
| 460 | 460_1 | CGgGGaAgCCttAC | 2.23 |
| 461 | 461_1 | CmcgGgGAagCCtTA | 1.59 |
| 462 | 462_1 | CCcGGgGAaGccTT | 1.32 |
| 463 | 463_1 | CccCGggGAaGcCT | 0.9 |
| 464 | 464_1 | ACccCGggGAagCC | 1.27 |
| 465 | 465_1 | CacCCcGggGAaGC | 0.78 |
| 466 | 466_1 | CCaCCccGgGgaAG | 0.76 |
| 467 | 467_1 | CCcAcCCmcgGggAA | 0.71 |
| 468 | 468_1 | CcCcAccCcGggGA | 0.67 |
| 469 | 469_1 | ACccCacCccGgGG | 0.79 |
| 470 | 470_1 | GAcCccAccCmcgGG | 0.78 |
| 471 | 471_1 | GgACccCacCccGG | 1.24 |
| 472 | 472_1 | AGgAcCccACccCG | 1.02 |
| 473 | 473_1 | GAggAccCcAccCC | 0.75 |
| 474 | 474_1 | GgaGgAccCCacCC | 0.51 |
| 475 | 475_1 | GggAGgAccCcaCC | 0.68 |
| 476 | 476_1 | TgGgAGgaCCccAC | 0.95 |
| 477 | 477_1 | CTggGAggACccCA | 0.46 |
| 478 | 478_1 | GgcTgGgaGgAcCC | 0.58 |
| 479 | 479_1 | AcGGctGGgAGgAC | 0.81 |
| 480 | 480_1 | CCacGGcTgGgaGG | 0.09 |
| 481 | 481_1 | GcCCacGgCTggGA | 0.52 |
| 482 | 482_1 | AGgCccAcGGctGG | 0.65 |
| 483 | 483_1 | TgAGgcCCacGgCT | 0.7 |
| 484 | 484_1 | CcTgaGGccCAcGG | 0.47 |
| 485 | 485_1 | AccCTgaGgCCcAC | 0.83 |
| 486 | 486_1 | TcAccCTgaGgcCC | 0.58 |
| 487 | 487_1 | GgtCacCctGagGC | 0.69 |
| 488 | 488_1 | TcGGtCAccCTgAG | 0.79 |
| 489 | 489_1 | GAtcGgTcaCCcTG | 0.72 |
| 490 | 490_1 | GTgATmcgGtCAcCC | 1.11 |
| 491 | 491_1 | CTgTGaTCgGTcAC | 0.69 |
| 492 | 492_1 | CCctGTgATcGgTC | 0.76 |
| 493 | 493_1 | CtCCctGTgATcGG | 0.56 |
| 494 | 494_1 | CTctCCctGTgaTC | 0.5 |
| 495 | 495_1 | CAcTctCCcTgtGA | 0.79 |
| 496 | 496_1 | GcCacTctCCctGT | 0.84 |
| 497 | 497_1 | GaGccACtCTccCT | 0.71 |
| 498 | 498_1 | GGgaGccAcTctCC | 0.65 |
| 499 | 499_1 | CAgGgaGcCActCT | 0.73 |
| 500 | 500_1 | GgcAGggAgcCaCT | 0.74 |
| 501 | 501_1 | AGggCagGgaGcCA | 0.62 |
| 502 | 502_1 | CcaGggCagGgaGC | 0.8 |
| 503 | 503_1 | GccCagGgcAGgGA | 0 |
| 504 | 504_1 | GTgCccAggGcaGG | 0.92 |
| 505 | 505_1 | GgGTgcCcaGggCA | 0 |
| 506 | 506_1 | GggGgtGccCagGG | 0 |
| 507 | 507_1 | CaGgGgGTgCccAG | 0.77 |
| 508 | 508_1 | CgCagGggGTgcCC | 0.12 |
| 509 | 509_1 | CCacCgCagGggGT | 0.82 |
| 510 | 510_1 | GggGCcAccGcaGG | 0 |
| 511 | 511_1 | TmcgGggCCacCgCA | 0.56 |
| 512 | 512_1 | CGtcGGggCCacCG | 0.86 |
| 513 | 513_1 | GTcGTcGggGCcAC | 0.65 |
| 514 | 514_1 | CTgTcGTmcgGggCC | 0.13 |
| 515 | 515_1 | TcaGCtGTcGTcGG | 0.77 |
| 516 | 516_1 | CCtcAGctGTmlcgTC | 0.54 |
| 517 | 517_1 | CTcCTcAGcTgtCG | 0.73 |
| 518 | 518_1 | CActCCtcAGctGT | 0.92 |
| 519 | 519_1 | GTcaCTccTCagCT | 1.25 |
| 520 | 520_1 | TgGTcaCTcCTcAG | 0.54 |
| 521 | 521_1 | TgTgGTcaCTccTC | 0.65 |
| 522 | 522_1 | CTtGTgGtcACtCC | 0.54 |
| 523 | 523_1 | ACcTTgTggTCaCT | 0.46 |
| 524 | 524_1 | AGacCTtGTgGtCA | 0.99 |
| 525 | 525_1 | AGaGAcCTtGTgGT | 0.8 |
| 526 | 526_1 | GCagAGacCTtgTG | 0.99 |
| 527 | 527_1 | GggCaGAgACctTG | 0.74 |
| 528 | 528_1 | GTgGgCAgaGacCT | 0.81 |
| 529 | 529_1 | CTgTgGgCaGAgAC | 0.74 |
| 530 | 530_1 | CActGTgGgCAgAG | 1.1 |
| 531 | 531_1 | AGcACtGTgGgcAG | 0.84 |
| 532 | 532_1 | CgAgCActGTggGC | 0.85 |
| 533 | 533_1 | CCcGAgCacTgtGG | 1.44 |
| 534 | 534_1 | ACcCCgaGcACtGT | 0.55 |
| 535 | 535_1 | GcAccCCgAGcaCT | 0.48 |
| 536 | 536_1 | CCgcAccCcGAgCA | 0.68 |
| 537 | 537_1 | CAccGCacCCmcgAG | 0.79 |
| 538 | 538_1 | GAcACcGcaCCcCG | 0.62 |
| 539 | 539_1 | CAgaCAccGCacCC | 0.77 |
| 540 | 540_1 | CCcaGAcaCCgcAC | 0.87 |
| 541 | 541_1 | AgcCCaGAcAccGC | 1.03 |
| 542 | 542_1 | GcaGccCAgAcaCC | 1.38 |
| 543 | 543_1 | TCgcAGccCAgaCA | 0.24 |
| 544 | 544_1 | CTtcGcAGcCCaGA | 0.35 |
| 545 | 545_1 | CAcTTcGCaGccCA | 0.9 |
| 546 | 546_1 | TcCacTTcGCagCC | 1.12 |
| 547 | 547_1 | GAtCCaCTtCgcAG | 0.49 |
| 548 | 548_1 | GGgATcCAcTtcGC | 1.19 |
| 549 | 549_1 | GGgGgATccACtTC | 0.59 |
| 550 | 550_1 | AaGgGgGAtCCaCT | 0.59 |
| 551 | 551_1 | GAaAGgGGgATcCA | 0.43 |
| 552 | 552_1 | AaGAaaGGgGGaTC | 0.5 |
| 553 | 553_1 | CCaAGaAAgGGgGA | 0.63 |
| 554 | 554_1 | GCcCaaGAaAGgGG | 0.98 |
| 555 | 555_1 | GTgCCcaAGaAaGG | 0.47 |
| 556 | 556_1 | CAgTGcCCaAGaAA | 0.52 |
| 557 | 557_1 | TgCaGTgCCcAaGA | 0.8 |
| 558 | 558_1 | GcTgCaGTgCCcAA | 0.99 |

The data, as illustrated in FIG. 1 illustrates that surprisingly antisense oligonucleotides targeting within the exon11 sequence are particularly effective in modulating the splicing of CARD9 pre-mRNA to enhance the production of the Δ11 CARD9 variant for example as compared to targeting the flanking intronic sequences or the intron/exon boundaries.

1. An antisense oligonucleotide for modulating the splicing of a mammalian CARD9 pre-mRNA transcript, wherein the oligonucleotide has a length of 10 to 40 nucleotides and comprises a contiguous nucleotide sequence of at least 10 nucleotides of at least 90% complementarity to SEQ ID NO 1.

2. The antisense oligonucleotide of claim 1, wherein the oligonucleotide has a length of 10-25 nucleotides.

3. (canceled)

4. The antisense oligonucleotide of claim 1, wherein the antisense oligonucleotide or contiguous nucleotide sequence thereof, is complementary to, such as fully complementary to any one of SEQ ID NOs: 6-9.

5-7. (canceled)

8. The antisense oligonucleotide of claim 1, wherein the contiguous nucleotide sequence comprises a nucleotide sequence which is complementary to, such as fully complementary to SEQ ID NO 559, 560, 561, 562, 563, 564 or 565.

9. The antisense oligonucleotide of claim 1, wherein the antisense oligonucleotide or contiguous nucleotide sequence thereof, is complementary to a sequence selected from the group consisting of SEQ ID NOs: 11-284.

10. The antisense oligonucleotide of claim 1 , wherein the antisense oligonucleotide is capable of enhancing the expression of Δ11CARD9 and/or reducing the expression of WTCARD9.

11-12. (canceled)

13. The antisense oligonucleotide of claim 1 , wherein the oligonucleotide is, or comprises an antisense oligonucleotide mixmer or totalmer.

14. The antisense oligonucleotide of claim 1, wherein the antisense oligonucleotide or contiguous nucleotide sequence thereof is 10 - 20 nucleotides in length.

15. The antisense oligonucleotide of claim 1, wherein the contiguous nucleotide sequence of the antisense oligonucleotide comprises a sequence selected from the group consisting of SEQ ID NOs: 285-558.

16. The antisense oligonucleotide of claim 1 , wherein the antisense oligonucleotide comprises an oligonucleotide provided herein, such as a compound selected from the group consisting of compound ID NO #285_1 -#558_1.

17. A conjugate comprising the antisense oligonucleotide according of claim 1, and at least one conjugate moiety covalently attached to the oligonucleotide.

18. A pharmaceutically acceptable salt of the antisense oligonucleotide to of claim 1, or the conjugate of claim 17.

19. A pharmaceutical composition comprising the antisense oligonucleotide of claim 1 or the conjugate of claim 17 and a pharmaceutically acceptable diluent, solvent, carrier, salt and/or adjuvant.

20. An in vivo or in vitro method for modulating the splicing of a CARD9 pre-mRNA in a mammalian target cell expressing CARD9, the method comprising administering an antisense oligonucleotide of claim 1, in an effective amount to the cell.

21. The method according to claim 20, wherein the administration of the antisense oligonucleotide results in enhanced expression of a CARD9Δ11 variant.

22. The method of claims 20 , wherein the antisense oligonucleotide results in reduced expression of WTCARD9.

23. (canceled)

24. The method of claim 20, wherein the CARD9 is a human CARD9.

25. A method for treating or preventing an inflammatory disease comprising administering a therapeutically or prophylactically effective amount of an antisense oligonucleotide of claims 1 to a subject suffering from or susceptible to the inflammatory disease, such as inflammatory bowel disease (such as Crohn’s disease and ulcerative colitis); or a disease selected from the group consisting of pancreatitis, IgA nephropathy, primary sclerosing cholangitis, cardiovascular disease, cancer and diabetes.

26-27. (canceled)

28. Use of the antisense oligonucleotide of claim 1 for the preparation of a medicament for treatment or prevention of an inflammatory disease, such as inflammatory bowel disease (such as Crohn’s disease and ulcerative colitis); or a disease selected from the group consisting of pancreatitis, IgA nephropathy, primary sclerosing cholangitis, cardiovascular disease, cancer and diabetes.


SEQUENCE LISTING (565 sequences)

SEQ ID NO: 1 - DNA, 9726 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcaggtgggtgggtctggtgctttctgaaaggtggccaggaaggttggcgttggcctagttcaggatcctggcactgatcccccagagcagcctgaggcttcccacaggcagccaggcagcctccgccatgcggccccgtccctttcacggcccctgggcggagatgtgttcatttcagtggaattacggtggcagatgggaccctgcatttcaagctggggaagcaggaaaggggcccagtgttcccaggactgtctaccgtgtggccagtgtttttattcagtcccttgtctgtcaaaactgtcctgaatggggtgacaaagtgcagcgggaagttgagaaatgcctcaaaagaccaagcctggggcaactatgtctccaggagcagtgacccagctcctctgggctggaaaccagcgtttcccagcggggctggaggctgtctgaggaccacgtggctgctcccagccttgccgcagtcccagtctcagagggagtggctttcagcggctgaggcccccaccatgctggcagaaagggacagcagtggctggtcccaacatgggtagttctgttgcagccactgggatgggggccgggcctcatctggggcaggagaagagtccagggattcactggtgcccggcacctcaacctggagaaaacaccgcagcagcactcctggccagccaagggcaacactgacagcccctcccagggttgcggggccggcagggggtgacagagacacgctcctgtgggcagctcttcccggccagtgcagcgaccccgcagctgcagcagggctgggtggccactcttgcatcatcagcagccggccgtggaggagggagggcacagccagggatgtgagcaggcgtcggtcaggggaatgaggaagtcccacaagaaccggctcccccagctgctgtgctggtgggagggccactggccaacctgctctcacccagcttgggctggccatagtgccaggcagttgccctcagctcctctgcccattccagccatggaggtggctaaccttgcaggctccagggcgtgatgggagtgtccaggaggcagaggccagagggggtcatgctgtagggggagttcctctctcagaccccatctgtgcctcccccgctgcgtatctgggtgacatggatgccaacgggcaggagcaagggctgggaaaggctgcggaggggccctcggctggtctggtggggagcggggtcactgtgtctctgctgcctccctgggggatccagcgtggggatgggacatggagctgggcacctctcctggatgcccctgggctgatccccctcgatgccctgacgagagccctggcccagcgtctgagaaggagtgggagctgggcccgggtgggggcaggagccggcatcgctctcagcactgccccacactccccagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtcagtgttgccccgcgggccccaggccccaaacccaaccacagagtcagctcggaggtggctgcacgctctgccaccctgaccctcaaccccaggagagccccgcctggctttgcccctggcctctgactgaggcccaacctcaaccctggcacgtggcccgtcccccagcctctcctgccggcttggtgctgccaagaggggcagcggggctcccaccgcagcgggggctcctgagacagccaaggggcgggtgggcagcacttggtccccagcctagtaccaagacccgcagggtacctcatccaagacccagaccacggcacacctcatctgcatgccagcctccacctgtccctgcccccaggcccacccaccccagccctctgcctcgcctgtgccccgcccccagttaggcctctctggcccgcaggtgtgctcctggacatcctgcagcggaccggccacaagggctacgtggccttcctcgagagcctggagctctactacccgcagctgtacaagaaggtcacaggcaaggagccggcccgcgtcttctccatgatcatcggtgagtgacgggggaggcggggccgacaggggaggcgctggcggggagcgggcagtggtgttgggtcctctgcctggcccccatccctaggagccctgagctcctgccacagcgcagggcgtgatggagctgggccaccggggaacacggcaccatctgacactggcctggcccacagacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggaggtgcgcccgctgcccccgggaaaccaggccaccctcgcctggcctggcggggtctgcaggccggggcggtcgggggtggccccacgggtctgcggtcaccgcctggaggggacagccaggctcactggaccttctgcagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggtggggccgtaggcagggcaggtgcagcccaggcagggcagtgggggccttcccagccaggccattgccttttgcccccttcaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccgggtaggagggcaggcggggagacatggcccctggggtccaagccaggctggacgggctgagctccgcccactccgtgcctggaaggtggggtgtggagaccccagtgcagcgcctccactcacccactcctggaggcacagtggacggtgccctctccttgcccgcctgccccccatggcaaaggccaacctctgggctcatggtgcctgaggcacagcagtgtctgaggcctgccggagggccctgtcctgccgcggtctggaagtgcagggctggtaggggcctggggactcctatggcctcccccgacatcccaactggcctgaccacatgcttctgagcccctgcccggagtataccccaagggccagaatggggggcctcaggcctgtgggggctgagctgccgccccctgcagtgcatggaggagaaggagatgttcgagctgcagtgcctggcactacgtaaggactccaagatgtacaaggaccgcatcgaggccatcctgctgcagatggaggaggtcgccattgagcgggaccaggtgggcactcgcaggggcaggacaaagcaccccactcgcgggtgggtgcaccagggccacggcccttgagggcagttgtcagaggatttttaatctttgttatttgtttttgagatggggtcttgctatgtcacccagggtggcctcaaactactggcctcaagcaatcctccggcctcagcctcccaaagtgctgggattgcaagctgagctgccacacctggtctgttttttaaatgacagaagcaatacttaacactaaaaaaaagtaaacgatgatgaagtcaatacttccccaccccagcctactgtcagggaaaaccgtgttcaaccttcccgctccaccttacagacttacctgtcatctatccatgtatccatctgtctatcatctatcacctatctatctatttatatatttggttttgttttacaaaaatggaaacattctgtcttgttttaccagtagctttttttaatcttaaaaaaagcttggctggacgtggtgtctctcgcctgtcatcccagcactttgggaggctgaggcgggcggatcacttgagcccaggagttccagagcagcctgggcaacacggtgagatccctgctctacaaaaaaatgcaaaaattggcctggcgcggtggctcacgcctataatcccagcactctaggagggcgaggcaggcggctcacttgaggtcaggagttcgagaccagcctggccagtatggggaaaccccgtctctactaaaaacacaaaaattagcctggtgtggttccgggcgcctgtaatcccagctactcaggaggctgaggcaggagaattgcttgaacccaggaggcagaggttgcagtgagctgagatcacgctactgtactccagcctgggtgacagagcgagactccgtctcaaaaaaaaaaaaaattaactggtcgtggtggcgcatgcctgtggtcccagctatttaggaggctgaggctgcagcaagtcaccattgcgctattgcactccagcctgggccacgggagtgaggcctgcaaggcctccagtgctacgtgctgtgagccggatggagcgtcgcggggcagggctgtgtctgcttcccatgccccgtcccaagtcccatcctggaggttgtttccaatggacaccattgctgcttcggctaaatgctggatggcactttgtccacatcaggagccttaaaacgagacccctggggctacaaaggaagccccggcccccatgagagcagcccaggctcagagctcctgtctcagccccacctcagcctcactcgcgcctggcccacgccaaggcaggggaaaagggcagatccagctgtgcccccatgggggttggtgtggctcccagggcgggggctcccacgggacttcccgggagcgcgttggccagggattagggaagctgccagccgttgctccagtctgggcagaaggggttaggtgagggcctcccagcctcggctgcaggggtggtcaagccgtcgtgtttgcacattcgctcctgcgtctcagtgtccgcagtgaggacaggggtggtaaagagtgctgcggtcacgaaggacgcttagctatggacagtcagggtctccatatcaagccgcgtccaaaatttatcaaatgtgcacgtgtgggggtacatagcacacgctgtgtccaccgggggtgcaggcttgggaggagacagaagggcagcagggacaactgtcacatttacaatcccccacgtgctgacttcgtgtccgcagagcacacaaatggaggggctgtgaccagcctccgcgcccagcggcttgacgtcctccggagcctctgcttggagttgggcggccgggccgagggcccagggcaagcttggggccctcactgagggtcggccttgtgctgtcccgtcaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctggtgggcctggggcctgcggcagggaggggagccggcgcgtgggatgcagggccaggggagcacggagcctcagccccggccttcccgacccaacccttctctgtgacgggccccaacctaaccaaacccctcctggcagtggggtgagggcacacgagcagttcagggtcccagcaggaagtggggctgcagggccggggtgggtcctgggcctggccatcaggcagcctagcaggttgttctgggcatggagggggcctggtgtggctgagggcatgcccagggctccctggaggatcccgctctgtgccctgcccaccctgtgcctggggagccctctgccctcacagcccacccaccccattttctatgaccacagagctccgacctggaagatggctcacccaggaggtcccaggaggtgagtgagaccccagccagcacgaggctggccttccttcacctggggtcctggctgagctgccaccaggcatagccccacactgcccgtcacatggccctgctcggtgggctgggctgggtggaggaagagcaggtggccaaatgctaaggcctctgatggctcaaggggtaaggtaaggcacggacagtgcccacctcctggccgcccaatgtccgtctgctgggcacctgagtctctgagaagctctgtgcagggggtgacggcaatggctgcacccagctgagggccaagggctggggtgggcagtgtggccgggtccagtccttggcgaagtggcctaatgggacatcttgactctctttggaagctctcactcccccaggacctggaggacacccagctctcagacaaaggtgaggtggggacagcagtacacccagaggcctcccagcttccccaagtgaggcccatcttcagcacaggcagcccgtgccgcaatgcctgtgtcaacccccacaacccccactccacagacagggaaactggggctggggataagtaaaatggtcaaggtcacctagagaacaaactacagagcccagctggaacccagggccccagctagtgggacaagtgtcccctggggtggagagtgtgggggtctgggcagcccccatgggctgcagaggtgagcaggcccaccgtcctgagagtgtggggcccctctgggcagcccccacggggtgcagaggtgagcaggcccagtcctggctgctccaagcttcctcctggacagagggaggtggtaacgagcgaacgaccaggtgcatgaagctagaagatggggcgagatggggccccacgtggcttgagggaaagggctgaatgcaggggtggccaagggcctgctgtgtcccggtgcagtgggggtaaggcagtggcaggggcttctagcgggcagggcaggccccctctgcctgtctgggggaaggactgggtggctagctgggagcagctcccgagtgtccaggagtgggcagtggtgatgaggtaggtgtttgcactgaaggtgctgtcaggtaggggttggggagggcccagcagaggggtcctgggaggctgcaccggagttggacaagcttcctgagtaccaggccgtggcctccaagcagaggcggaaggggaaggaggagccgcagacggggaagtgaagaccagcgcctttggctgcagctcagaacacttgaacatggggatgccaagcccccatgtaggcacagaggcgagcaggacccaggagtccggctcaggcactgcccccatccccctgggtggctgtggcaggagcaggggcaggtgtgaaggttggagggtgagcaggggaggggctggctctaaggggctgccaatggccaggacagccctggcctggccctcaccgcagtggggctgagactggacgtgcgtggtttctgcccaagggtcctgatccctggtctgcccaggtcctggcggtttctgagggggtccagccgcctgaaggttctctcccaattgctgcagtacccctgcccccacttcaggaggacccagacagccactgtagctgtccccaactctctccccagggccgccatgcccacagatgctttggagtgatgggggacggtgctgctcaggcccagggtttaagttcccccgaggtctgggaggcgttcctccctggcaggcagccagctcgggggagggatggtcactggcgggagcgttgggcctgggctggcatctggcggcctcctggagcaatgaacaggcaagcgaggccataaggcctcagtctcatctacagaaagcagagtctacactggacccatgtggggatcccaccaggccttggaagacagccccactgcaatgccggggaggggtggggcagccgtgctatacgtggcccagcagatgggcaggtcccacaggcacgactctcctttccaggctgccttgccggcggggggagcccgaaacagccctttgcagctctgcaccaggagcaggttttgcggaacccccatgtaaggcttccccggggtggggtcctcccagccgtgggcctcagggtgaccgatcacagggagagtggctccctgccctgggcaccccctgcggtggccccgacgacagctgaggagtgaccacaaggtctctgcccacagtgctcggggtgcggtgtctgggctgcgaagtggatccccctttcttgggcactgcagcagcttggggggctttttggacgtggatgtgcctggtcctggttttccgagggcctttacagtggatgaggaggtgaacacaggagtcctgagagcaagcaccacctcgggctttgttgtagaaacaatggcccggaccccaggctggagccgtggcttggcctcctgggtgtgtcttggcatctgaaatgcaggctacccacaccggctcacctccaggggtacaggcaggtcccacagggagagcttggcgctgagctgaggctgtctgggctcctcgcctcccaaccagtctgcagttacaggggccagtggggggcgggtgagaaggacgggttccctcaggggagccggccggagcccgagccttcccccttctccaggacgcaggcctgagcagcggggagccgcccgagaaggagcggcggcgcctcaaagagagttttgagaactaccgcaggtaggcgggcggcccccaggcttctccaaaacgggctggggagcggcagccgggtcgcgcctgagacacctgtcttcacaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgca

SEQ ID NO: 2 - DNA, 2147 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggctgccttgccggcggggggagcccgaaacagccctttgcagctctgcaccaggagcaggttttgcggaacccccatgacgcaggcctgagcagcggggagccgcccgagaaggagcggcggcgcctcaaagagagttttgagaactaccgcaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgcaaaaaaaaaaaaaaaaaaaaaaaaa

SEQ ID NO: 3 - protein, 536 aa, homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGCLAGGGSPKQPFAALHQEQVLRNPHDAGLSSGEPPEKERRRLKESFENYRRKRALRKMQKGWRQGEEDRENTTGSDNTDTEGS

SEQ ID NO: 4 - DNA, 2070 bp, homo sapiens
gactgtggagttaagcagaacccatcaggaagtgcacaggcgtccggcgtgctcctccctccctgcagccccgggcagcatctcccagaggctccgcggcccaggctcctggtgtgtctgcagtgcaggtggctcctggaagaccctcagcctgcctgctgaggccatgtcggactacgagaacgatgacgagtgctggagcgtcctggagggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtgtgctcctggacatcctgcagcggaccggccacaagggctacgtggccttcctcgagagcctggagctctactacccgcagctgtacaagaaggtcacaggcaaggagccggcccgcgtcttctccatgatcatcgacgcgtccggggagtcaggcctgactcagctgctgatgactgaggtcatgaagctgcagaagaaggtgcaggacctgaccgcgctgctgagctccaaagatgacttcatcaaggagctgcgggtgaaggacagcctgctgcgcaagcaccaggagcgtgtgcagaggctcaaggaggagtgcgaggccggcagccgcgagctcaagcgctgcaaggaggagaactacgacctggccatgcgcctggcgcaccagagtgaggagaagggcgccgcgctcatgcggaaccgtgacctgcagctggagattgaccagctcaagcacagcctcatgaaggccgaggacgactgcaaggtggagcgcaagcacacgctgaagctcaggcacgccatggagcagcggcccagccaggagctgctgtgggagctgcagcaggagaaggccctgctccaggcccgggtgcaggagctggaggcctccgtccaggaggggaagctggacaggagcagcccctacatccaggtactggaggaggactggcggcaggcgctgcgggaccaccaggagcaggccaacaccatcttctccctgcgcaaggacctccgccagggcgaggcccgacgcctccggtgcatggaggagaaggagatgttcgagctgcagtgcctggcactacgtaaggactccaagatgtacaaggaccgcatcgaggccatcctgctgcagatggaggaggtcgccattgagcgggaccaggccatagccacgcgggaggagctgcacgcacagcacgcccggggcctgcaggagaaggacgcgctgcgcaagcaggtgcgggagctgggcgagaaggcggatgagctgcagctgcaggtgttccagtgtgaggcgcagctactggccgtggagggcaggctcaggcggcagcagctggagacgctcgtcctgagctccgacctggaagatggctcacccaggaggtcccaggagctctcactcccccaggacctggaggacacccagctctcagacaaaggacgcaggcctgagcagcggggagccgcccgagaaggagcggcggcgcctcaaagagagttttgagaactaccgcaggaagcgcgccctcaggaagatgcagaaaggatggcggcagggggaggaggaccgggagaacaccacgggcagcgacaacaccgacactgagggctcctagccgcagcagcgcaggccccgaccagggcacacccaccggcccggcctcctgccacccgggggtgccgacgccctggggcgcagacttccccgagccgtcgctgacttggcctggaacgaggaatctggtgccctgaaaggcccagccggactgccgggcattggggccgtttgttaagcggcactcattttgcggaggccatgcgggtgctcaccacccccatgcacacgccatctgtgtaacttcaggatctgttctgtttcaccatgtaacacacaatacatgcatgcattgtattagtgttagaaaacacagctgcgtaaataaacagcacgggtgacccgcaaaaaaaaaaaaaaaaaaaaaaaaa

SEQ ID NO: 5 - protein, 473 aa, homo sapiens
MSDYENDDECWSVLEGFRVTLTSVIDPSRITPYLRQCKVLNPDDEEQVLSDPNLVIRKRKVGVLLDILQRTGHKGYVAFLESLELYYPQLYKKVTGKEPARVFSMIIDASGESGLTQLLMTEVMKLQKKVQDLTALLSSKDDFIKELRVKDSLLRKHQERVQRLKEECEAGSRELKRCKEENYDLAMRLAHQSEEKGAALMRNRDLQLEIDQLKHSLMKAEDDCKVERKHTLKLRHAMEQRPSQELLWELQQEKALLQARVQELEASVQEGKLDRSSPYIQVLEEDWRQALRDHQEQANTIFSLRKDLRQGEARRLRCMEEKEMFELQCLALRKDSKMYKDRIEAILLQMEEVAIERDQAIATREELHAQHARGLQEKDALRKQVRELGEKADELQLQVFQCEAQLLAVEGRLRRQQLETLVLSSDLEDGSPRRSQELSLPQDLEDTQLSDKGRRPEQRGAAREGAAAPQREF

SEQ ID NO: 6 - DNA, 476 bp, homo sapiens
tcctggagcaatgaacaggcaagcgaggccataaggcctcagtctcatctacagaaagcagagtctacactggacccatgtggggatcccaccaggccttggaagacagccccactgcaatgccggggaggggtggggcagccgtgctatacgtggcccagcagatgggcaggtcccacaggcacgactctcctttccaggctgccttgccggcggggggagcccgaaacagccctttgcagctctgcaccaggagcaggttttgcggaacccccatgtaaggcttccccggggtggggtcctcccagccgtgggcctcagggtgaccgatcacagggagagtggctccctgccctgggcaccccctgcggtggccccgacgacagctgaggagtgaccacaaggtctctgcccacagtgctcggggtgcggtgtctgggctgcgaagtggatccccctttcttgggcactgcagc

SEQ ID NO: 7 - DNA, 77 bp, homo sapiens
gctgccttgccggcggggggagcccgaaacagccctttgcagctctgcaccaggagcaggttttgcggaacccccat

SEQ ID NO: 8 - DNA, 49 bp, homo sapiens
gcagctctgcaccaggagcaggttttgcggaacccccatgtaaggcttc

SEQ ID NO: 9 - DNA, 59 bp, homo sapiens
cctttgcagctctgcaccaggagcaggttttgcggaacccccatgtaaggcttccccgg

SEQ ID NO: 10 - protein, 25 aa, homo sapiens
CLAGGGSPKQPFAALHQEQVLRNPH

SEQ ID NO: 11 - DNA, 14 bp, homo sapiens
tcctggagcaatga

SEQ ID NO: 12 - DNA, 14 bp, homo sapiens
ctggagcaatgaac

SEQ ID NO: 13 - DNA, 14 bp, homo sapiens
ggagcaatgaacag

SEQ ID NO: 14 - DNA, 14 bp, homo sapiens
agcaatgaacaggc

SEQ ID NO: 15 - DNA, 14 bp, homo sapiens
caatgaacaggcaa

SEQ ID NO: 16 - DNA, 14 bp, homo sapiens
atgaacaggcaagc

SEQ ID NO: 17 - DNA, 14 bp, homo sapiens
gaacaggcaagcga

SEQ ID NO: 18 - DNA, 14 bp, homo sapiens
acaggcaagcgagg

SEQ ID NO: 19 - DNA, 14 bp, homo sapiens
aggcaagcgaggcc

SEQ ID NO: 20 - DNA, 14 bp, homo sapiens
gcaagcgaggccat

SEQ ID NO: 21 - DNA, 14 bp, homo sapiens
aagcgaggccataa

SEQ ID NO: 22 - DNA, 14 bp, homo sapiens
gcgaggccataagg

SEQ ID NO: 23 - DNA, 14 bp, homo sapiens
gaggccataaggcc

SEQ ID NO: 24 - DNA, 14 bp, homo sapiens
ggccataaggcctc

SEQ ID NO: 25 - DNA, 14 bp, homo sapiens
ccataaggcctcag

SEQ ID NO: 26 - DNA, 14 bp, homo sapiens
ataaggcctcagtc

SEQ ID NO: 27 - DNA, 14 bp, homo sapiens
aaggcctcagtctc

SEQ ID NO: 28 - DNA, 14 bp, homo sapiens
ggcctcagtctcat

SEQ ID NO: 29 - DNA, 14 bp, homo sapiens
cctcagtctcatct

SEQ ID NO: 30 - DNA, 14 bp, homo sapiens
tcagtctcatctac

SEQ ID NO: 31 - DNA, 14 bp, homo sapiens
agtctcatctacag

SEQ ID NO: 32 - DNA, 14 bp, homo sapiens
tctcatctacagaa

SEQ ID NO: 33 - DNA, 14 bp, homo sapiens
tcatctacagaaag

SEQ ID NO: 34 - DNA, 14 bp, homo sapiens
atctacagaaagca

SEQ ID NO: 35 - DNA, 14 bp, homo sapiens
ctacagaaagcaga

SEQ ID NO: 36 - DNA, 14 bp, homo sapiens
acagaaagcagagt

SEQ ID NO: 37 - DNA, 14 bp, homo sapiens
agaaagcagagtct

SEQ ID NO: 38 - DNA, 14 bp, homo sapiens
aaagcagagtctac

SEQ ID NO: 39 - DNA, 14 bp, homo sapiens
agcagagtctacac

SEQ ID NO: 40 - DNA, 14 bp, homo sapiens
cagagtctacactg

SEQ ID NO: 41 - DNA, 14 bp, homo sapiens
gagtctacactgga

SEQ ID NO: 42 - DNA, 14 bp, homo sapiens
gtctacactggacc

SEQ ID NO: 43 - DNA, 14 bp, homo sapiens
ctacactggaccca

SEQ ID NO: 44 - DNA, 14 bp, homo sapiens
acactggacccatg

SEQ ID NO: 45 - DNA, 14 bp, homo sapiens
actggacccatgtg

SEQ ID NO: 46 - DNA, 14 bp, homo sapiens
tggacccatgtggg

SEQ ID NO: 47 - DNA, 14 bp, homo sapiens
gacccatgtgggga

SEQ ID NO: 48 - DNA, 14 bp, homo sapiens
cccatgtggggatc

SEQ ID NO: 49 - DNA, 14 bp, homo sapiens
catgtggggatccc

SEQ ID NO: 50 - DNA, 14 bp, homo sapiens
tgtggggatcccac

SEQ ID NO: 51 - DNA, 14 bp, homo sapiens
tggggatcccacca

SEQ ID NO: 52 - DNA, 14 bp, homo sapiens
gggatcccaccagg

SEQ ID NO: 53 - DNA, 14 bp, homo sapiens
gatcccaccaggcc

SEQ ID NO: 54 - DNA, 14 bp, homo sapiens
tcccaccaggcctt

SEQ ID NO: 55 - DNA, 14 bp, homo sapiens
ccaccaggccttgg

SEQ ID NO: 56 - DNA, 14 bp, homo sapiens
accaggccttggaa

SEQ ID NO: 57 - DNA, 14 bp, homo sapiens
caggccttggaaga

SEQ ID NO: 58 - DNA, 14 bp, homo sapiens
ggccttggaagaca

SEQ ID NO: 59 - DNA, 14 bp, homo sapiens
ccttggaagacagc

SEQ ID NO: 60 - DNA, 14 bp, homo sapiens
ttggaagacagccc

SEQ ID NO: 61 - DNA, 14 bp, homo sapiens
ggaagacagcccca

SEQ ID NO: 62 - DNA, 14 bp, homo sapiens
aagacagccccact

SEQ ID NO: 63 - DNA, 14 bp, homo sapiens
gacagccccactgc

SEQ ID NO: 64 - DNA, 14 bp, homo sapiens
cagccccactgcaa

SEQ ID NO: 65 - DNA, 14 bp, homo sapiens
gccccactgcaatg

SEQ ID NO: 66 - DNA, 14 bp, homo sapiens
cccactgcaatgcc

SEQ ID NO: 67 - DNA, 14 bp, homo sapiens
cactgcaatgccgg

SEQ ID NO: 68 - DNA, 14 bp, homo sapiens
ctgcaatgccgggg

SEQ ID NO: 69 - DNA, 14 bp, homo sapiens
gcaatgccggggag

SEQ ID NO: 70 - DNA, 14 bp, homo sapiens
aatgccggggaggg

SEQ ID NO: 71 - DNA, 14 bp, homo sapiens
tgccggggaggggt

SEQ ID NO: 72 - DNA, 14 bp, homo sapiens
ccggggaggggtgg

SEQ ID NO: 73 - DNA, 14 bp, homo sapiens
ggggaggggtgggg

SEQ ID NO: 74 - DNA, 14 bp, homo sapiens
ggaggggtggggca

SEQ ID NO: 75 - DNA, 14 bp, homo sapiens
aggggtggggcagc

SEQ ID NO: 76 - DNA, 14 bp, homo sapiens
gggtggggcagccg

SEQ ID NO: 77 - DNA, 14 bp, homo sapiens
gtggggcagccgtg

SEQ ID NO: 78 - DNA, 14 bp, homo sapiens
ggggcagccgtgct

SEQ ID NO: 79 - DNA, 14 bp, homo sapiens
ggcagccgtgctat

SEQ ID NO: 80 - DNA, 14 bp, homo sapiens
cagccgtgctatac

SEQ ID NO: 81 - DNA, 14 bp, homo sapiens
gccgtgctatacgt

SEQ ID NO: 82 - DNA, 14 bp, homo sapiens
ccgtgctatacgtg

SEQ ID NO: 83 - DNA, 14 bp, homo sapiens
cgtgctatacgtgg

SEQ ID NO: 84 - DNA, 14 bp, homo sapiens
gtgctatacgtggc

SEQ ID NO: 85 - DNA, 14 bp, homo sapiens
tgctatacgtggcc

SEQ ID NO: 86 - DNA, 14 bp, homo sapiens
gctatacgtggccc

SEQ ID NO: 87 - DNA, 14 bp, homo sapiens
ctatacgtggccca

SEQ ID NO: 88 - DNA, 14 bp, homo sapiens
tatacgtggcccag

SEQ ID NO: 89 - DNA, 14 bp, homo sapiens
atacgtggcccagc

SEQ ID NO: 90 - DNA, 14 bp, homo sapiens
tacgtggcccagca

SEQ ID NO: 91 - DNA, 14 bp, homo sapiens
acgtggcccagcag

SEQ ID NO: 92 - DNA, 14 bp, homo sapiens
cgtggcccagcaga

SEQ ID NO: 93 - DNA, 14 bp, homo sapiens
gtggcccagcagat

SEQ ID NO: 94 - DNA, 14 bp, homo sapiens
tggcccagcagatg

SEQ ID NO: 95 - DNA, 14 bp, homo sapiens
ggcccagcagatgg

SEQ ID NO: 96 - DNA, 14 bp, homo sapiens
gcccagcagatggg

SEQ ID NO: 97 - DNA, 14 bp, homo sapiens
cccagcagatgggc

SEQ ID NO: 98 - DNA, 14 bp, homo sapiens
ccagcagatgggca

SEQ ID NO: 99 - DNA, 14 bp, homo sapiens
cagcagatgggcag

SEQ ID NO: 100 - DNA, 14 bp, homo sapiens
agcagatgggcagg

SEQ ID NO: 101 - DNA, 14 bp, homo sapiens
gcagatgggcaggt

SEQ ID NO: 102 - DNA, 14 bp, homo sapiens
cagatgggcaggtc

SEQ ID NO: 103 - DNA, 14 bp, homo sapiens
agatgggcaggtcc

SEQ ID NO: 104 - DNA, 14 bp, homo sapiens
gatgggcaggtccc

SEQ ID NO: 105 - DNA, 14 bp, homo sapiens
atgggcaggtccca

SEQ ID NO: 106 - DNA, 14 bp, homo sapiens
tgggcaggtcccac

SEQ ID NO: 107 - DNA, 14 bp, homo sapiens
gggcaggtcccaca

SEQ ID NO: 108 - DNA, 14 bp, homo sapiens
ggcaggtcccacag

SEQ ID NO: 109 - DNA, 14 bp, homo sapiens
gcaggtcccacagg

SEQ ID NO: 110 - DNA, 14 bp, homo sapiens
caggtcccacaggc

SEQ ID NO: 111 - DNA, 14 bp, homo sapiens
aggtcccacaggca

SEQ ID NO: 112 - DNA, 14 bp, homo sapiens
ggtcccacaggcac

SEQ ID NO: 113 - DNA, 14 bp, homo sapiens
gtcccacaggcacg

SEQ ID NO: 114 - DNA, 14 bp, homo sapiens
tcccacaggcacga

SEQ ID NO: 115 - DNA, 14 bp, homo sapiens
cccacaggcacgac

SEQ ID NO: 116 - DNA, 14 bp, homo sapiens
ccacaggcacgact

SEQ ID NO: 117 - DNA, 14 bp, homo sapiens
cacaggcacgactc

SEQ ID NO: 118 - DNA, 14 bp, homo sapiens
acaggcacgactct

SEQ ID NO: 119 - DNA, 14 bp, homo sapiens
caggcacgactctc

SEQ ID NO: 120 - DNA, 14 bp, homo sapiens
aggcacgactctcc

SEQ ID NO: 121 - DNA, 14 bp, homo sapiens
ggcacgactctcct

SEQ ID NO: 122 - DNA, 14 bp, homo sapiens
gcacgactctcctt

SEQ ID NO: 123 - DNA, 14 bp, homo sapiens
cacgactctccttt

SEQ ID NO: 124 - DNA, 14 bp, homo sapiens
acgactctcctttc

SEQ ID NO: 125 - DNA, 14 bp, homo sapiens
cgactctcctttcc

SEQ ID NO: 126 - DNA, 14 bp, homo sapiens
gactctcctttcca

SEQ ID NO: 127 - DNA, 14 bp, homo sapiens
actctcctttccag

SEQ ID NO: 128 - DNA, 14 bp, homo sapiens
ctctcctttccagg

SEQ ID NO: 129 - DNA, 14 bp, homo sapiens
tctcctttccaggc

SEQ ID NO: 130 - DNA, 14 bp, homo sapiens
ctcctttccaggct

SEQ ID NO: 131 - DNA, 14 bp, homo sapiens
tcctttccaggctg

SEQ ID NO: 132 - DNA, 14 bp, homo sapiens
cctttccaggctgc

SEQ ID NO: 133 - DNA, 14 bp, homo sapiens
ctttccaggctgcc

SEQ ID NO: 134 - DNA, 14 bp, homo sapiens
tttccaggctgcct

SEQ ID NO: 135 - DNA, 14 bp, homo sapiens
ttccaggctgcctt

SEQ ID NO: 136 - DNA, 14 bp, homo sapiens
tccaggctgccttg

SEQ ID NO: 137 - DNA, 14 bp, homo sapiens
ccaggctgccttgc

SEQ ID NO: 138 - DNA, 14 bp, homo sapiens
caggctgccttgcc

SEQ ID NO: 139 - DNA, 14 bp, homo sapiens
aggctgccttgccg

SEQ ID NO: 140 - DNA, 14 bp, homo sapiens
ggctgccttgccgg

SEQ ID NO: 141 - DNA, 14 bp, homo sapiens
gctgccttgccggc

SEQ ID NO: 142 - DNA, 14 bp, homo sapiens
ctgccttgccggcg

SEQ ID NO: 143 - DNA, 14 bp, homo sapiens
tgccttgccggcgg

SEQ ID NO: 144 - DNA, 14 bp, homo sapiens
gccttgccggcggg

SEQ ID NO: 145 - DNA, 14 bp, homo sapiens
ccttgccggcgggg

SEQ ID NO: 146 - DNA, 14 bp, homo sapiens
cttgccggcggggg

SEQ ID NO: 147 - DNA, 14 bp, homo sapiens
ttgccggcgggggg

SEQ ID NO: 148 - DNA, 14 bp, homo sapiens
tgccggcgggggga

SEQ ID NO: 149 - DNA, 14 bp, homo sapiens
gccggcggggggag

SEQ ID NO: 150 - DNA, 14 bp, homo sapiens
ccggcggggggagc

SEQ ID NO: 151 - DNA, 14 bp, homo sapiens
cggcggggggagcc

SEQ ID NO: 152 - DNA, 14 bp, homo sapiens
ggcggggggagccc

SEQ ID NO: 153 - DNA, 14 bp, homo sapiens
gcggggggagcccg

SEQ ID NO: 154 - DNA, 14 bp, homo sapiens
cggggggagcccga

SEQ ID NO: 155 - DNA, 14 bp, homo sapiens
ggggggagcccgaa

SEQ ID NO: 156 - DNA, 14 bp, homo sapiens
gggggagcccgaaa

SEQ ID NO: 157 - DNA, 14 bp, homo sapiens
ggggagcccgaaac

SEQ ID NO: 158 - DNA, 14 bp, homo sapiens
gggagcccgaaaca

SEQ ID NO: 159 - DNA, 14 bp, homo sapiens
ggagcccgaaacag

SEQ ID NO: 160 - DNA, 14 bp, homo sapiens
gagcccgaaacagc

SEQ ID NO: 161 - DNA, 14 bp, homo sapiens
agcccgaaacagcc

SEQ ID NO: 162 - DNA, 14 bp, homo sapiens
gcccgaaacagccc

SEQ ID NO: 163 - DNA, 14 bp, homo sapiens
cccgaaacagccct

SEQ ID NO: 164 - DNA, 14 bp, homo sapiens
ccgaaacagccctt

SEQ ID NO: 165 - DNA, 14 bp, homo sapiens
cgaaacagcccttt

SEQ ID NO: 166 - DNA, 14 bp, homo sapiens
gaaacagccctttg

SEQ ID NO: 167 - DNA, 14 bp, homo sapiens
aaacagccctttgc

SEQ ID NO: 168 - DNA, 14 bp, homo sapiens
aacagccctttgca

SEQ ID NO: 169 - DNA, 14 bp, homo sapiens
acagccctttgcag

SEQ ID NO: 170 - DNA, 14 bp, homo sapiens
cagccctttgcagc

SEQ ID NO: 171 - DNA, 14 bp, homo sapiens
agccctttgcagct

SEQ ID NO: 172 - DNA, 14 bp, homo sapiens
gccctttgcagctc

SEQ ID NO: 173 - DNA, 14 bp, homo sapiens
ccctttgcagctct

SEQ ID NO: 174 - DNA, 14 bp, homo sapiens
cctttgcagctctg

SEQ ID NO: 175 - DNA, 14 bp, homo sapiens
tgcagctctgcacc

SEQ ID NO: 176 - DNA, 14 bp, homo sapiens
gctctgcaccagga

SEQ ID NO: 177 - DNA, 14 bp, homo sapiens
tgcaccaggagcag

SEQ ID NO: 178 - DNA, 14 bp, homo sapiens
ccaggagcaggttt

SEQ ID NO: 179 - DNA, 14 bp, homo sapiens
gagcaggttttgcg

SEQ ID NO: 180 - DNA, 14 bp, homo sapiens
aggttttgcggaac

SEQ ID NO: 181 - DNA, 14 bp, homo sapiens
ttgcggaaccccca

SEQ ID NO: 182 - DNA, 14 bp, homo sapiens
ggaacccccatgta

SEQ ID NO: 183 - DNA, 14 bp, homo sapiens
cccccatgtaaggc

SEQ ID NO: 184 - DNA, 14 bp, homo sapiens
catgtaaggcttcc

SEQ ID NO: 185 - DNA, 14 bp, homo sapiens
tgtaaggcttcccc

SEQ ID NO: 186 - DNA, 14 bp, homo sapiens
gtaaggcttccccg

SEQ ID NO: 187 - DNA, 14 bp, homo sapiens
taaggcttccccgg

SEQ ID NO: 188 - DNA, 14 bp, homo sapiens
aaggcttccccggg

SEQ ID NO: 189 - DNA, 14 bp, homo sapiens
aggcttccccgggg

SEQ ID NO: 190 - DNA, 14 bp, homo sapiens
ggcttccccggggt

SEQ ID NO: 191 - DNA, 14 bp, homo sapiens
gcttccccggggtg

SEQ ID NO: 192 - DNA, 14 bp, homo sapiens
cttccccggggtgg

SEQ ID NO: 193 - DNA, 14 bp, homo sapiens
ttccccggggtggg

SEQ ID NO: 194 - DNA, 14 bp, homo sapiens
tccccggggtgggg

SEQ ID NO: 195 - DNA, 14 bp, homo sapiens
ccccggggtggggt

SEQ ID NO: 196 - DNA, 14 bp, homo sapiens
cccggggtggggtc

SEQ ID NO: 197 - DNA, 14 bp, homo sapiens
ccggggtggggtcc

SEQ ID NO: 198 - DNA, 14 bp, homo sapiens
cggggtggggtcct

SEQ ID NO: 199 - DNA, 14 bp, homo sapiens
ggggtggggtcctc

SEQ ID NO: 200 - DNA, 14 bp, homo sapiens
gggtggggtcctcc

SEQ ID NO: 201 - DNA, 14 bp, homo sapiens
ggtggggtcctccc

SEQ ID NO: 202 - DNA, 14 bp, homo sapiens
gtggggtcctccca

SEQ ID NO: 203 - DNA, 14 bp, homo sapiens
tggggtcctcccag

SEQ ID NO: 204 - DNA, 14 bp, homo sapiens
gggtcctcccagcc

SEQ ID NO: 205 - DNA, 14 bp, homo sapiens
gtcctcccagccgt

SEQ ID NO: 206 - DNA, 14 bp, homo sapiens
cctcccagccgtgg

SEQ ID NO: 207 - DNA, 14 bp, homo sapiens
tcccagccgtgggc

SEQ ID NO: 208 - DNA, 14 bp, homo sapiens
ccagccgtgggcct

SEQ ID NO: 209 - DNA, 14 bp, homo sapiens
agccgtgggcctca

SEQ ID NO: 210 - DNA, 14 bp, homo sapiens
ccgtgggcctcagg

SEQ ID NO: 211 - DNA, 14 bp, homo sapiens
gtgggcctcagggt

SEQ ID NO: 212 - DNA, 14 bp, homo sapiens
gggcctcagggtga

SEQ ID NO: 213 - DNA, 14 bp, homo sapiens
gcctcagggtgacc

SEQ ID NO: 214 - DNA, 14 bp, homo sapiens
ctcagggtgaccga

SEQ ID NO: 215 - DNA, 14 bp, homo sapiens
cagggtgaccgatc

SEQ ID NO: 216 - DNA, 14 bp, homo sapiens
gggtgaccgatcac

SEQ ID NO: 217 - DNA, 14 bp, homo sapiens
gtgaccgatcacag

SEQ ID NO: 218 - DNA, 14 bp, homo sapiens
gaccgatcacaggg

SEQ ID NO: 219 - DNA, 14 bp, homo sapiens
ccgatcacagggag

SEQ ID NO: 220 - DNA, 14 bp, homo sapiens
gatcacagggagag

SEQ ID NO: 221 - DNA, 14 bp, homo sapiens
tcacagggagagtg

SEQ ID NO: 222 - DNA, 14 bp, homo sapiens
acagggagagtggc

SEQ ID NO: 223 - DNA, 14 bp, homo sapiens
agggagagtggctc

SEQ ID NO: 224 - DNA, 14 bp, homo sapiens
ggagagtggctccc

SEQ ID NO: 225 - DNA, 14 bp, homo sapiens
agagtggctccctg

SEQ ID NO: 226 - DNA, 14 bp, homo sapiens
agtggctccctgcc

SEQ ID NO: 227 - DNA, 14 bp, homo sapiens
tggctccctgccct

SEQ ID NO: 228 - DNA, 14 bp, homo sapiens
gctccctgccctgg

SEQ ID NO: 229 - DNA, 14 bp, homo sapiens
tccctgccctgggc

SEQ ID NO: 230 - DNA, 14 bp, homo sapiens
cctgccctgggcac

SEQ ID NO: 231 - DNA, 14 bp, homo sapiens
tgccctgggcaccc

SEQ ID NO: 232 - DNA, 14 bp, homo sapiens
ccctgggcaccccc

SEQ ID NO: 233 - DNA, 14 bp, homo sapiens
ctgggcaccccctg

SEQ ID NO: 234 - DNA, 14 bp, homo sapiens
gggcaccccctgcg

SEQ ID NO: 235 - DNA, 14 bp, homo sapiens
accccctgcggtgg

SEQ ID NO: 236 - DNA, 14 bp, homo sapiens
cctgcggtggcccc

SEQ ID NO: 237 - DNA, 14 bp, homo sapiens
tgcggtggccccga

SEQ ID NO: 238 - DNA, 14 bp, homo sapiens
cggtggccccgacg

SEQ ID NO: 239 - DNA, 14 bp, homo sapiens
gtggccccgacgac

SEQ ID NO: 240 - DNA, 14 bp, homo sapiens
ggccccgacgacag

SEQ ID NO: 241 - DNA, 14 bp, homo sapiens
ccgacgacagctga

SEQ ID NO: 242 - DNA, 14 bp, homo sapiens
gacgacagctgagg

SEQ ID NO: 243 - DNA, 14 bp, homo sapiens
cgacagctgaggag

SEQ ID NO: 244 - DNA, 14 bp, homo sapiens
acagctgaggagtg

SEQ ID NO: 245 - DNA, 14 bp, homo sapiens
agctgaggagtgac

SEQ ID NO: 246 - DNA, 14 bp, homo sapiens
ctgaggagtgacca

SEQ ID NO: 247 - DNA, 14 bp, homo sapiens
gaggagtgaccaca

SEQ ID NO: 248 - DNA, 14 bp, homo sapiens
ggagtgaccacaag

SEQ ID NO: 249 - DNA, 14 bp, homo sapiens
agtgaccacaaggt

SEQ ID NO: 250 - DNA, 14 bp, homo sapiens
tgaccacaaggtct

SEQ ID NO: 251 - DNA, 14 bp, homo sapiens
accacaaggtctct

SEQ ID NO: 252 - DNA, 14 bp, homo sapiens
cacaaggtctctgc

SEQ ID NO: 253 - DNA, 14 bp, homo sapiens
caaggtctctgccc

SEQ ID NO: 254 - DNA, 14 bp, homo sapiens
aggtctctgcccac

SEQ ID NO: 255 - DNA, 14 bp, homo sapiens
gtctctgcccacag

SEQ ID NO: 256 - DNA, 14 bp, homo sapiens
ctctgcccacagtg

SEQ ID NO: 257 - DNA, 14 bp, homo sapiens
ctgcccacagtgct

SEQ ID NO: 258 - DNA, 14 bp, homo sapiens
gcccacagtgctcg

SEQ ID NO: 259 - DNA, 14 bp, homo sapiens
ccacagtgctcggg

SEQ ID NO: 260 - DNA, 14 bp, homo sapiens
acagtgctcggggt

SEQ ID NO: 261 - DNA, 14 bp, homo sapiens
agtgctcggggtgc

SEQ ID NO: 262 - DNA, 14 bp, homo sapiens
tgctcggggtgcgg

SEQ ID NO: 263 - DNA, 14 bp, homo sapiens
ctcggggtgcggtg

SEQ ID NO: 264 - DNA, 14 bp, homo sapiens
cggggtgcggtgtc

SEQ ID NO: 265 - DNA, 14 bp, homo sapiens
gggtgcggtgtctg

SEQ ID NO: 266 - DNA, 14 bp, homo sapiens
gtgcggtgtctggg

SEQ ID NO: 267 - DNA, 14 bp, homo sapiens
gcggtgtctgggct

SEQ ID NO: 268 - DNA, 14 bp, homo sapiens
ggtgtctgggctgc

SEQ ID NO: 269 - DNA, 14 bp, homo sapiens
tgtctgggctgcga

SEQ ID NO: 270 - DNA, 14 bp, homo sapiens
tctgggctgcgaag

SEQ ID NO: 271 - DNA, 14 bp, homo sapiens
tgggctgcgaagtg

SEQ ID NO: 272 - DNA, 14 bp, homo sapiens
ggctgcgaagtgga

SEQ ID NO: 273 - DNA, 14 bp, homo sapiens
ctgcgaagtggatc

SEQ ID NO: 274 - DNA, 14 bp, homo sapiens
gcgaagtggatccc

SEQ ID NO: 275 - DNA, 14 bp, homo sapiens
gaagtggatccccc

SEQ ID NO: 276 - DNA, 14 bp, homo sapiens
agtggatccccctt

SEQ ID NO: 277 - DNA, 14 bp, homo sapiens
tggatccccctttc

SEQ ID NO: 278 - DNA, 14 bp, homo sapiens
gatccccctttctt

SEQ ID NO: 279 - DNA, 14 bp, homo sapiens
tccccctttcttgg

SEQ ID NO: 280 - DNA, 14 bp, homo sapiens
cccctttcttgggc

SEQ ID NO: 281 - DNA, 14 bp, homo sapiens
cctttcttgggcac

SEQ ID NO: 282 - DNA, 14 bp, homo sapiens
tttcttgggcactg

SEQ ID NO: 283 - DNA, 14 bp, homo sapiens
tcttgggcactgca

SEQ ID NO: 284 - DNA, 14 bp, homo sapiens
ttgggcactgcagc

SEQ ID NO: 285 - DNA, 14 bp, Artificial Sequence
  Oligonucleotide nucleobase sequence motif
tcattgctccagga

SEQ ID NO: 286 - DNA, 14 bp, Artificial Sequence
  Oligonucleotide nucleobase sequence motif
gttcattgctccag

SEQ ID NO: 287 - DNA, 14 bp, Artificial Sequence
  Oligonucleotide nucleobase sequence motif
ctgttcattgctcc

SEQ ID NO: 288 - DNA, 14 bp, Artificial Sequence
  Oligonucleotide nucleobase sequence motif
gcctgttcattgct

SEQ ID NO: 289 - DNA, 14 bp, Artificial Sequence
  Oligonucleotide nucleobase sequence motif
ttgcctgttcattg

SEQ ID NO: 290 - DNA, 14 bp, Artificial Sequence
  Oligonucleotide nucleobase sequence motif
gcttgcctgttcat

SEQ ID NO: 291 - DNA, 14 bp, Artificial Sequence
  Oligonucleotide nucleobase sequence motif
tcgcttgcctgttc

SEQ ID NO: 292 - DNA, 14 bp, Artificial Sequence
  Oligonucleotide nucleobase sequence motif
cctcgcttgcctgt

SEQ ID NO: 293 - DNA, 14 bp, Artificial Sequence
  Oligonucleotide nucleobase sequence motif
ggcctcgcttgcct

SEQ ID NO: 294 - DNA, 14 bp, Artificial Sequence
  Oligonucleotide nucleobase sequence motif
atggcctcgcttgc

SEQ ID NO: 295 - DNA, 14 bp, Artificial Sequence
  Oligonucleotide nucleobase sequence motif
ttatggcctcgctt

SEQ ID NO: 296 - DNA, 14 bp, Artificial Sequence
  Oligonucleotide nucleobase sequence motif
ccttatggcctcgc

SEQ ID NO: 297 - DNA, 14 bp, Artificial Sequence
  Oligonucleotide nucleobase sequence motif
ggccttatggcctc

SEQ ID NO: 298 - DNA, 14 bp, Artificial Sequence
  Oligonucleotide nucleobase sequence motif
gaggccttatggcc

SEQ ID NO: 299 - DNA, 14 bp, Artificial Sequence
  Oligonucleotide nucleobase sequence motif
ctgaggccttatgg

SEQ ID NO: 300 - DNA, 14 bp, Artificial Sequence
  Oligonucleotide nucleobase sequence motif
gactgaggccttat

SEQ ID NO: 301 - DNA, 14 bp, Artificial Sequence
  Oligonucleotide nucleobase sequence motif
gagactgaggcctt

SEQ ID NO: 302 - DNA, 14 bp, Artificial Sequence
  Oligonucleotide nucleobase sequence motif
atgagactgaggcc

SEQ ID NO: 303 - DNA, 14 bp, Artificial Sequence
  Oligonucleotide nucleobase sequence motif
agatgagactgagg

SEQ ID NO: 304 - DNA, 14 bp, Artificial Sequence
  Oligonucleotide nucleobase sequence motif
gtagatgagactga

SEQ ID NO: 305 - DNA, 14 bp, Artificial Sequence
  Oligonucleotide nucleobase sequence motif
ctgtagatgagact

SEQ ID NO: 306 - DNA, 14 bp, Artificial Sequence
  Oligonucleotide nucleobase sequence motif
ttctgtagatgaga

SEQ ID NO: 307 - DNA, 14 bp, Artificial Sequence
  Oligonucleotide nucleobase sequence motif
ctttctgtagatga

SEQ ID NO: 308 - DNA, 14 bp, Artificial Sequence
  Oligonucleotide nucleobase sequence motif
tgctttctgtagat

SEQ ID NO: 309 - DNA, 14 bp, Artificial Sequence
  Oligonucleotide nucleobase sequence motif
tctgctttctgtag

SEQ ID NO: 310 - DNA, 14 bp, Artificial Sequence
  Oligonucleotide nucleobase sequence motif
actctgctttctgt

SEQ ID NO: 311 - DNA, 14 bp, Artificial Sequence
  Oligonucleotide nucleobase sequence motif
agactctgctttct

SEQ ID NO: 312 - DNA, 14 bp, Artificial Sequence
  Oligonucleotide nucleobase sequence motif
gtagactctgcttt

SEQ ID NO: 313 - DNA, 14 bp, Artificial Sequence
  Oligonucleotide nucleobase sequence motif
gtgtagactctgct

SEQ ID NO: 314 - DNA, 14 bp, Artificial Sequence
  Oligonucleotide nucleobase sequence motif
cagtgtagactctg

SEQ ID NO: 315 - DNA, 14 bp, Artificial Sequence
  Oligonucleotide nucleobase sequence motif
tccagtgtagactc

SEQ ID NO: 316 - DNA, 14 bp, Artificial Sequence
  Oligonucleotide nucleobase sequence motif
ggtccagtgtagac

SEQ ID NO: 317 - DNA, 14 bp, Artificial Sequence
  Oligonucleotide nucleobase sequence motif
tgggtccagtgtag

SEQ ID NO: 318 - DNA, 14 bp, Artificial Sequence
  Oligonucleotide nucleobase sequence motif
catgggtccagtgt

SEQ ID NO: 319 - DNA, 14 bp, Artificial Sequence
  Oligonucleotide nucleobase sequence motif
cacatgggtccagt

SEQ ID NO: 320 - DNA, 14 bp, Artificial Sequence
  Oligonucleotide nucleobase sequence motif
cccacatgggtcca

SEQ ID NO: 321 - DNA, 14 bp, Artificial Sequence
  Oligonucleotide nucleobase sequence motif
tccccacatgggtc

SEQ ID NO: 322 - DNA, 14 bp, Artificial Sequence
  Oligonucleotide nucleobase sequence motif
gatccccacatggg

SEQ ID NO: 323 - DNA, 14 bp, Artificial Sequence
  Oligonucleotide nucleobase sequence motif
gggatccccacatg

SEQ ID NO: 324 - DNA, 14 bp, Artificial Sequence
  Oligonucleotide nucleobase sequence motif
gtgggatccccaca

SEQ ID NO: 325 - DNA, 14 bp, Artificial Sequence
  Oligonucleotide nucleobase sequence motif
tggtgggatcccca

SEQ ID NO: 326 - DNA, 14 bp, Artificial Sequence
  Oligonucleotide nucleobase sequence motif
cctggtgggatccc

SEQ ID NO: 327 - DNA, 14 bp, Artificial Sequence
  Oligonucleotide nucleobase sequence motif
ggcctggtgggatc

SEQ ID NO: 328 - DNA, 14 bp, Artificial Sequence
  Oligonucleotide nucleobase sequence motif
aaggcctggtggga

SEQ ID NO: 329 - DNA, 14 bp, Artificial Sequence
  Oligonucleotide nucleobase sequence motif
ccaaggcctggtgg

SEQ ID NO: 330 - DNA, 14 bp, Artificial Sequence
  Oligonucleotide nucleobase sequence motif
ttccaaggcctggt

SEQ ID NO: 331 - DNA, 14 bp, Artificial Sequence
  Oligonucleotide nucleobase sequence motif
tcttccaaggcctg

SEQ ID NO: 332 - DNA, 14 bp, Artificial Sequence
  Oligonucleotide nucleobase sequence motif
tgtcttccaaggcc

SEQ ID NO: 333 - DNA, 14 bp, Artificial Sequence
  Oligonucleotide nucleobase sequence motif
gctgtcttccaagg

SEQ ID NO: 334 - DNA, 14 bp, Artificial Sequence
  Oligonucleotide nucleobase sequence motif
gggctgtcttccaa

SEQ ID NO: 335 - DNA, 14 bp, Artificial Sequence
  Oligonucleotide nucleobase sequence motif
tggggctgtcttcc

SEQ ID NO: 336 - DNA, 14 bp, Artificial Sequence
  Oligonucleotide nucleobase sequence motif
agtggggctgtctt

SEQ ID NO: 337 - DNA, 14 bp, Artificial Sequence
  Oligonucleotide nucleobase sequence motif
gcagtggggctgtc

SEQ ID NO: 338 - DNA, 14 bp, Artificial Sequence
  Oligonucleotide nucleobase sequence motif
ttgcagtggggctg

SEQ ID NO: 339 - DNA, 14 bp, Artificial Sequence
  Oligonucleotide nucleobase sequence motif
cattgcagtggggc

SEQ ID NO: 340 - DNA, 14 bp, Artificial Sequence
  Oligonucleotide nucleobase sequence motif
ggcattgcagtggg

SEQ ID NO: 341 - DNA, 14 bp, Artificial Sequence
  Oligonucleotide nucleobase sequence motif
ccggcattgcagtg

SEQ ID NO: 342 - DNA, 14 bp, Artificial Sequence
  Oligonucleotide nucleobase sequence motif
ccccggcattgcag

SEQ ID NO: 343 - DNA, 14 bp, Artificial Sequence
  Oligonucleotide nucleobase sequence motif
ctccccggcattgc

SEQ ID NO: 344 - DNA, 14 bp, Artificial Sequence
  Oligonucleotide nucleobase sequence motif
ccctccccggcatt

SEQ ID NO: 345 - DNA, 14 bp, Artificial Sequence
  Oligonucleotide nucleobase sequence motif
acccctccccggca

SEQ ID NO: 346 - DNA, 14 bp, Artificial Sequence
  Oligonucleotide nucleobase sequence motif
ccacccctccccgg

SEQ ID NO: 347 - DNA, 14 bp, Artificial Sequence
  Oligonucleotide nucleobase sequence motif
ccccacccctcccc

SEQ ID NO: 348 - DNA, 14 bp, Artificial Sequence
  Oligonucleotide nucleobase sequence motif
tgccccacccctcc

SEQ ID NO: 349 - DNA, 14 bp, Artificial Sequence
  Oligonucleotide nucleobase sequence motif
gctgccccacccct

SEQ ID NO: 350 - DNA, 14 bp, Artificial Sequence
  Oligonucleotide nucleobase sequence motif
cggctgccccaccc

SEQ ID NO: 351 - DNA, 14 bp, Artificial Sequence
  Oligonucleotide nucleobase sequence motif
cacggctgccccac

SEQ ID NO: 352 - DNA, 14 bp, Artificial Sequence
  Oligonucleotide nucleobase sequence motif
agcacggctgcccc

SEQ ID NO: 353 - DNA, 14 bp, Artificial Sequence
  Oligonucleotide nucleobase sequence motif
atagcacggctgcc

SEQ ID NO: 354 - DNA, 14 bp, Artificial Sequence
  Oligonucleotide nucleobase sequence motif
gtatagcacggctg

SEQ ID NO: 355 - DNA, 14 bp, Artificial Sequence
  Oligonucleotide nucleobase sequence motif
acgtatagcacggc

SEQ ID NO: 356 - DNA, 14 bp, Artificial Sequence
  Oligonucleotide nucleobase sequence motif
cacgtatagcacgg

SEQ ID NO: 357 - DNA, 14 bp, Artificial Sequence
  Oligonucleotide nucleobase sequence motif
ccacgtatagcacg

SEQ ID NO: 358 - DNA, 14 bp, Artificial Sequence
  Oligonucleotide nucleobase sequence motif
gccacgtatagcac

SEQ ID NO: 359 - DNA, 14 bp, Artificial Sequence
  Oligonucleotide nucleobase sequence motif
ggccacgtatagca

SEQ ID NO: 360 - DNA, 14 bp, Artificial Sequence
  Oligonucleotide nucleobase sequence motif
gggccacgtatagc

SEQ ID NO: 361 - DNA, 14 bp, Artificial Sequence
  Oligonucleotide nucleobase sequence motif
tgggccacgtatag

SEQ ID NO: 362 - DNA, 14 bp, Artificial Sequence
  Oligonucleotide nucleobase sequence motif
ctgggccacgtata

SEQ ID NO: 363 - DNA, 14 bp, Artificial Sequence
  Oligonucleotide nucleobase sequence motif
gctgggccacgtat

SEQ ID NO: 364 - DNA, 14 bp, Artificial Sequence
  Oligonucleotide nucleobase sequence motif
tgctgggccacgta

SEQ ID NO: 365 - DNA, 14 bp, Artificial Sequence
  Oligonucleotide nucleobase sequence motif
ctgctgggccacgt

SEQ ID NO: 366 - DNA, 14 bp, Artificial Sequence
  Oligonucleotide nucleobase sequence motif
tctgctgggccacg

SEQ ID NO: 367 - DNA, 14 bp, Artificial Sequence
  Oligonucleotide nucleobase sequence motif
atctgctgggccac

SEQ ID NO: 368 - DNA, 14 bp, Artificial Sequence
  Oligonucleotide nucleobase sequence motif
catctgctgggcca

SEQ ID NO: 369 - DNA, 14 bp, Artificial Sequence
  Oligonucleotide nucleobase sequence motif
ccatctgctgggcc

SEQ ID NO: 370 - DNA, 14 bp, Artificial Sequence
  Oligonucleotide nucleobase sequence motif
cccatctgctgggc

SEQ ID NO: 371 - DNA, 14 bp, Artificial Sequence
  Oligonucleotide nucleobase sequence motif
gcccatctgctggg

SEQ ID NO: 372 - DNA, 14 bp, Artificial Sequence
  Oligonucleotide nucleobase sequence motif
tgcccatctgctgg

SEQ ID NO: 373 - DNA, 14 bp, Artificial Sequence
  Oligonucleotide nucleobase sequence motif
ctgcccatctgctg

SEQ ID NO: 374 - DNA, 14 bp, Artificial Sequence
  Oligonucleotide nucleobase sequence motif
cctgcccatctgct

SEQ ID NO: 375 - DNA, 14 bp, Artificial Sequence
  Oligonucleotide nucleobase sequence motif
acctgcccatctgc

SEQ ID NO: 376 - DNA, 14 bp, Artificial Sequence
  Oligonucleotide nucleobase sequence motif
gacctgcccatctg

SEQ ID NO: 377 - DNA, 14 bp, Artificial Sequence
  Oligonucleotide nucleobase sequence motif
ggacctgcccatct

SEQ ID NO: 378 - DNA, 14 bp, Artificial Sequence
  Oligonucleotide nucleobase sequence motif
gggacctgcccatc

SEQ ID NO: 379 - DNA, 14 bp, Artificial Sequence
  Oligonucleotide nucleobase sequence motif
tgggacctgcccat

SEQ ID NO: 380 - DNA, 14 bp, Artificial Sequence
  Oligonucleotide nucleobase sequence motif
gtgggacctgccca

SEQ ID NO: 381 - DNA, 14 bp, Artificial Sequence
  Oligonucleotide nucleobase sequence motif
tgtgggacctgccc

SEQ ID NO: 382 - DNA, 14 bp, Artificial Sequence
  Oligonucleotide nucleobase sequence motif
ctgtgggacctgcc

SEQ ID NO: 383 - DNA, 14 bp, Artificial Sequence
  Oligonucleotide nucleobase sequence motif
cctgtgggacctgc

SEQ ID NO: 384 - DNA, 14 bp, Artificial Sequence
  Oligonucleotide nucleobase sequence motif
gcctgtgggacctg

SEQ ID NO: 385 - DNA, 14 bp, Artificial Sequence
  Oligonucleotide nucleobase sequence motif
tgcctgtgggacct

SEQ ID NO: 386 - DNA, 14 bp, Artificial Sequence
  Oligonucleotide nucleobase sequence motif
gtgcctgtgggacc

SEQ ID NO: 387 - DNA, 14 bp, Artificial Sequence
  Oligonucleotide nucleobase sequence motif
cgtgcctgtgggac

SEQ ID NO: 388 - DNA, 14 bp, Artificial Sequence
  Oligonucleotide nucleobase sequence motif
tcgtgcctgtggga

SEQ ID NO: 389 - DNA, 14 bp, Artificial Sequence
  Oligonucleotide nucleobase sequence motif
gtcgtgcctgtggg

SEQ ID NO: 390 - DNA, 14 bp, Artificial Sequence
  Oligonucleotide nucleobase sequence motif
agtcgtgcctgtgg

SEQ ID NO: 391 - DNA, 14 bp, Artificial Sequence
  Oligonucleotide nucleobase sequence motif
gagtcgtgcctgtg

SEQ ID NO: 392 - DNA, 14 bp, Artificial Sequence
  Oligonucleotide nucleobase sequence motif
agagtcgtgcctgt

SEQ ID NO: 393 - DNA, 14 bp, Artificial Sequence
  Oligonucleotide nucleobase sequence motif
gagagtcgtgcctg

SEQ ID NO: 394 - DNA, 14 bp, Artificial Sequence
  Oligonucleotide nucleobase sequence motif
ggagagtcgtgcct

SEQ ID NO: 395 - DNA, 14 bp, Artificial Sequence
  Oligonucleotide nucleobase sequence motif
aggagagtcgtgcc

SEQ ID NO: 396 - DNA, 14 bp, Artificial Sequence
  Oligonucleotide nucleobase sequence motif
aaggagagtcgtgc

SEQ ID NO: 397 - DNA, 14 bp, Artificial Sequence
  Oligonucleotide nucleobase sequence motif
aaaggagagtcgtg

SEQ ID NO: 398 - DNA, 14 bp, Artificial Sequence
  Oligonucleotide nucleobase sequence motif
gaaaggagagtcgt

SEQ ID NO: 399 - DNA, 14 bp, Artificial Sequence
  Oligonucleotide nucleobase sequence motif
ggaaaggagagtcg

SEQ ID NO: 400 - DNA, 14 bp, Artificial Sequence
  Oligonucleotide nucleobase sequence motif
tggaaaggagagtc

SEQ ID NO: 401 - DNA, 14 bp, Artificial Sequence
  Oligonucleotide nucleobase sequence motif
ctggaaaggagagt

SEQ ID NO: 402 - DNA, 14 bp, Artificial Sequence
  Oligonucleotide nucleobase sequence motif
cctggaaaggagag

SEQ ID NO: 403 - DNA, 14 bp, Artificial Sequence
  Oligonucleotide nucleobase sequence motif
gcctggaaaggaga

SEQ ID NO: 404 - DNA, 14 bp, Artificial Sequence
  Oligonucleotide nucleobase sequence motif
agcctggaaaggag

SEQ ID NO: 405 - DNA, 14 bp, Artificial Sequence
  Oligonucleotide nucleobase sequence motif
cagcctggaaagga

SEQ ID NO: 406 - DNA, 14 bp, Artificial Sequence
  Oligonucleotide nucleobase sequence motif
gcagcctggaaagg

SEQ ID NO: 407 - DNA, 14 bp, Artificial Sequence
  Oligonucleotide nucleobase sequence motif
ggcagcctggaaag

SEQ ID NO: 408 - DNA, 14 bp, Artificial Sequence
  Oligonucleotide nucleobase sequence motif
aggcagcctggaaa

SEQ ID NO: 409 - DNA, 14 bp, Artificial Sequence
  Oligonucleotide nucleobase sequence motif
aaggcagcctggaa

SEQ ID NO: 410 - DNA, 14 bp, Artificial Sequence
  Oligonucleotide nucleobase sequence motif
caaggcagcctgga

SEQ ID NO: 411 - DNA, 14 bp, Artificial Sequence
  Oligonucleotide nucleobase sequence motif
gcaaggcagcctgg

SEQ ID NO: 412 - DNA, 14 bp, Artificial Sequence
  Oligonucleotide nucleobase sequence motif
ggcaaggcagcctg

SEQ ID NO: 413 - DNA, 14 bp, Artificial Sequence
  Oligonucleotide nucleobase sequence motif
cggcaaggcagcct

SEQ ID NO: 414 - DNA, 14 bp, Artificial Sequence
  Oligonucleotide nucleobase sequence motif
ccggcaaggcagcc

SEQ ID NO: 415 - DNA, 14 bp, Artificial Sequence
  Oligonucleotide nucleobase sequence motif
gccggcaaggcagc

SEQ ID NO: 416 - DNA, 14 bp, Artificial Sequence
  Oligonucleotide nucleobase sequence motif
cgccggcaaggcag

SEQ ID NO: 417 - DNA, 14 bp, Artificial Sequence
  Oligonucleotide nucleobase sequence motif
ccgccggcaaggca

SEQ ID NO: 418 - DNA, 14 bp, Artificial Sequence
  Oligonucleotide nucleobase sequence motif
cccgccggcaaggc

SEQ ID NO: 419 - DNA, 14 bp, Artificial Sequence
  Oligonucleotide nucleobase sequence motif
ccccgccggcaagg

SEQ ID NO: 420 - DNA, 14 bp, Artificial Sequence
  Oligonucleotide nucleobase sequence motif
cccccgccggcaag

SEQ ID NO: 421 - DNA, 14 bp, Artificial Sequence
  Oligonucleotide nucleobase sequence motif
ccccccgccggcaa

SEQ ID NO: 422 - DNA, 14 bp, Artificial Sequence
  Oligonucleotide nucleobase sequence motif
tccccccgccggca

SEQ ID NO: 423 - DNA, 14 bp, Artificial Sequence
  Oligonucleotide nucleobase sequence motif
ctccccccgccggc

SEQ ID NO: 424 - DNA, 14 bp, Artificial Sequence
  Oligonucleotide nucleobase sequence motif
gctccccccgccgg

SEQ ID NO: 425 - DNA, 14 bp, Artificial Sequence
  Oligonucleotide nucleobase sequence motif
ggctccccccgccg

SEQ ID NO: 426 - DNA, 14 bp, Artificial Sequence
  Oligonucleotide nucleobase sequence motif
gggctccccccgcc

SEQ ID NO: 427 - DNA, 14 bp, Artificial Sequence
  Oligonucleotide nucleobase sequence motif
cgggctccccccgc

SEQ ID NO: 428 - DNA, 14 bp, Artificial Sequence
  Oligonucleotide nucleobase sequence motif
tcgggctccccccg

SEQ ID NO: 429 - DNA, 14 bp, Artificial Sequence
  Oligonucleotide nucleobase sequence motif
ttcgggctcccccc

SEQ ID NO: 430 - DNA, 14 bp, Artificial Sequence
  Oligonucleotide nucleobase sequence motif
tttcgggctccccc

SEQ ID NO: 431 - DNA, 14 bp, Artificial Sequence
  Oligonucleotide nucleobase sequence motif
gtttcgggctcccc

SEQ ID NO: 432 - DNA, 14 bp, Artificial Sequence
  Oligonucleotide nucleobase sequence motif
tgtttcgggctccc

SEQ ID NO: 433 - DNA, 14 bp, Artificial Sequence
  Oligonucleotide nucleobase sequence motif
ctgtttcgggctcc

SEQ ID NO: 434 - DNA, 14 bp, Artificial Sequence
  Oligonucleotide nucleobase sequence motif
gctgtttcgggctc

SEQ ID NO: 435 - DNA, 14 bp, Artificial Sequence
  Oligonucleotide nucleobase sequence motif
ggctgtttcgggct

SEQ ID NO: 436 - DNA, 14 bp, Artificial Sequence
  Oligonucleotide nucleobase sequence motif
gggctgtttcgggc

SEQ ID NO: 437 - DNA, 14 bp, Artificial Sequence
  Oligonucleotide nucleobase sequence motif
agggctgtttcggg

SEQ ID NO: 438 - DNA, 14 bp, Artificial Sequence
  Oligonucleotide nucleobase sequence motif
aagggctgtttcgg

SEQ ID NO: 439 - DNA, 14 bp, Artificial Sequence
  Oligonucleotide nucleobase sequence motif
aaagggctgtttcg

SEQ ID NO: 440 - DNA, 14 bp, Artificial Sequence
  Oligonucleotide nucleobase sequence motif
caaagggctgtttc

SEQ ID NO: 441 - DNA, 14 bp, Artificial Sequence
  Oligonucleotide nucleobase sequence motif
gcaaagggctgttt

SEQ ID NO: 442 - DNA, 14 bp, Artificial Sequence
  Oligonucleotide nucleobase sequence motif
tgcaaagggctgtt

SEQ ID NO: 443 - DNA, 14 bp, Artificial Sequence
  Oligonucleotide nucleobase sequence motif
ctgcaaagggctgt

SEQ ID NO: 444 - DNA, 14 bp, Artificial Sequence
  Oligonucleotide nucleobase sequence motif
gctgcaaagggctg

SEQ ID NO: 445 - DNA, 14 bp, Artificial Sequence
  Oligonucleotide nucleobase sequence motif
agctgcaaagggct

SEQ ID NO: 446 - DNA, 14 bp, Artificial Sequence
  Oligonucleotide nucleobase sequence motif
gagctgcaaagggc

SEQ ID NO: 447 - DNA, 14 bp, Artificial Sequence
  Oligonucleotide nucleobase sequence motif
agagctgcaaaggg

SEQ ID NO: 448 - DNA, 14 bp, Artificial Sequence
  Oligonucleotide nucleobase sequence motif
cagagctgcaaagg

SEQ ID NO: 449 - DNA, 14 bp, Artificial Sequence
  Oligonucleotide nucleobase sequence motif
ggtgcagagctgca

SEQ ID NO: 450 - DNA, 14 bp, Artificial Sequence
  Oligonucleotide nucleobase sequence motif
tcctggtgcagagc

SEQ ID NO: 451 - DNA, 14 bp, Artificial Sequence
  Oligonucleotide nucleobase sequence motif
ctgctcctggtgca

SEQ ID NO: 452 - DNA, 14 bp, Artificial Sequence
  Oligonucleotide nucleobase sequence motif
aaacctgctcctgg

SEQ ID NO: 453 - DNA, 14 bp, Artificial Sequence
  Oligonucleotide nucleobase sequence motif
cgcaaaacctgctc

SEQ ID NO: 454 - DNA, 14 bp, Artificial Sequence
  Oligonucleotide nucleobase sequence motif
gttccgcaaaacct

SEQ ID NO: 455 - DNA, 14 bp, Artificial Sequence
  Oligonucleotide nucleobase sequence motif
tgggggttccgcaa

SEQ ID NO: 456 - DNA, 14 bp, Artificial Sequence
  Oligonucleotide nucleobase sequence motif
tacatgggggttcc

SEQ ID NO: 457 - DNA, 14 bp, Artificial Sequence
  Oligonucleotide nucleobase sequence motif
gccttacatggggg

SEQ ID NO: 458 - DNA, 14 bp, Artificial Sequence
  Oligonucleotide nucleobase sequence motif
ggaagccttacatg

SEQ ID NO: 459 - DNA, 14 bp, Artificial Sequence
  Oligonucleotide nucleobase sequence motif
ggggaagccttaca

SEQ ID NO: 460 - DNA, 14 bp, Artificial Sequence
  Oligonucleotide nucleobase sequence motif
cggggaagccttac

SEQ ID NO: 461 - DNA, 14 bp, Artificial Sequence
  Oligonucleotide nucleobase sequence motif
ccggggaagcctta

SEQ ID NO: 462 - DNA, 14 bp, Artificial Sequence
  Oligonucleotide nucleobase sequence motif
cccggggaagcctt

SEQ ID NO: 463 - DNA, 14 bp, Artificial Sequence
  Oligonucleotide nucleobase sequence motif
ccccggggaagcct

SEQ ID NO: 464 - DNA, 14 bp, Artificial Sequence
  Oligonucleotide nucleobase sequence motif
accccggggaagcc

SEQ ID NO: 465 - DNA, 14 bp, Artificial Sequence
  Oligonucleotide nucleobase sequence motif
caccccggggaagc

SEQ ID NO: 466 - DNA, 14 bp, Artificial Sequence
  Oligonucleotide nucleobase sequence motif
ccaccccggggaag

SEQ ID NO: 467 - DNA, 14 bp, Artificial Sequence
  Oligonucleotide nucleobase sequence motif
cccaccccggggaa

SEQ ID NO: 468 - DNA, 14 bp, Artificial Sequence
  Oligonucleotide nucleobase sequence motif
ccccaccccgggga

SEQ ID NO: 469 - DNA, 14 bp, Artificial Sequence
  Oligonucleotide nucleobase sequence motif
accccaccccgggg

SEQ ID NO: 470 - DNA, 14 bp, Artificial Sequence
  Oligonucleotide nucleobase sequence motif
gaccccaccccggg

SEQ ID NO: 471 - DNA, 14 bp, Artificial Sequence
  Oligonucleotide nucleobase sequence motif
ggaccccaccccgg

SEQ ID NO: 472 - DNA, 14 bp, Artificial Sequence
  Oligonucleotide nucleobase sequence motif
aggaccccaccccg

SEQ ID NO: 473 - DNA, 14 bp, Artificial Sequence
  Oligonucleotide nucleobase sequence motif
gaggaccccacccc

SEQ ID NO: 474 - DNA, 14 bp, Artificial Sequence
  Oligonucleotide nucleobase sequence motif
ggaggaccccaccc

SEQ ID NO: 475 - DNA, 14 bp, Artificial Sequence
  Oligonucleotide nucleobase sequence motif
gggaggaccccacc

SEQ ID NO: 476 - DNA, 14 bp, Artificial Sequence
  Oligonucleotide nucleobase sequence motif
tgggaggaccccac

SEQ ID NO: 477 - DNA, 14 bp, Artificial Sequence
  Oligonucleotide nucleobase sequence motif
ctgggaggacccca

SEQ ID NO: 478 - DNA, 14 bp, Artificial Sequence
  Oligonucleotide nucleobase sequence motif
ggctgggaggaccc

SEQ ID NO: 479 - DNA, 14 bp, Artificial Sequence
  Oligonucleotide nucleobase sequence motif
acggctgggaggac

SEQ ID NO: 480 - DNA, 14 bp, Artificial Sequence
  Oligonucleotide nucleobase sequence motif
ccacggctgggagg

SEQ ID NO: 481 - DNA, 14 bp, Artificial Sequence
  Oligonucleotide nucleobase sequence motif
gcccacggctggga

SEQ ID NO: 482 - DNA, 14 bp, Artificial Sequence
  Oligonucleotide nucleobase sequence motif
aggcccacggctgg

SEQ ID NO: 483 - DNA, 14 bp, Artificial Sequence
  Oligonucleotide nucleobase sequence motif
tgaggcccacggct

SEQ ID NO: 484 - DNA, 14 bp, Artificial Sequence
  Oligonucleotide nucleobase sequence motif
cctgaggcccacgg

SEQ ID NO: 485 - DNA, 14 bp, Artificial Sequence
  Oligonucleotide nucleobase sequence motif
accctgaggcccac

SEQ ID NO: 486 - DNA, 14 bp, Artificial Sequence
  Oligonucleotide nucleobase sequence motif
tcaccctgaggccc

SEQ ID NO: 487 - DNA, 14 bp, Artificial Sequence
  Oligonucleotide nucleobase sequence motif
ggtcaccctgaggc

SEQ ID NO: 488 - DNA, 14 bp, Artificial Sequence
  Oligonucleotide nucleobase sequence motif
tcggtcaccctgag

SEQ ID NO: 489 - DNA, 14 bp, Artificial Sequence
  Oligonucleotide nucleobase sequence motif
gatcggtcaccctg

SEQ ID NO: 490 - DNA, 14 bp, Artificial Sequence
  Oligonucleotide nucleobase sequence motif
gtgatcggtcaccc

SEQ ID NO: 491 - DNA, 14 bp, Artificial Sequence
  Oligonucleotide nucleobase sequence motif
ctgtgatcggtcac

SEQ ID NO: 492 - DNA, 14 bp, Artificial Sequence
  Oligonucleotide nucleobase sequence motif
ccctgtgatcggtc

SEQ ID NO: 493 - DNA, 14 bp, Artificial Sequence
  Oligonucleotide nucleobase sequence motif
ctccctgtgatcgg

SEQ ID NO: 494 - DNA, 14 bp, Artificial Sequence
  Oligonucleotide nucleobase sequence motif
ctctccctgtgatc

SEQ ID NO: 495 - DNA, 14 bp, Artificial Sequence
  Oligonucleotide nucleobase sequence motif
cactctccctgtga

SEQ ID NO: 496 - DNA, 14 bp, Artificial Sequence
  Oligonucleotide nucleobase sequence motif
gccactctccctgt

SEQ ID NO: 497 - DNA, 14 bp, Artificial Sequence
  Oligonucleotide nucleobase sequence motif
gagccactctccct

SEQ ID NO: 498 - DNA, 14 bp, Artificial Sequence
  Oligonucleotide nucleobase sequence motif
gggagccactctcc

SEQ ID NO: 499 - DNA, 14 bp, Artificial Sequence
  Oligonucleotide nucleobase sequence motif
cagggagccactct

SEQ ID NO: 500 - DNA, 14 bp, Artificial Sequence
  Oligonucleotide nucleobase sequence motif
ggcagggagccact

SEQ ID NO: 501 - DNA, 14 bp, Artificial Sequence
  Oligonucleotide nucleobase sequence motif
agggcagggagcca

SEQ ID NO: 502 - DNA, 14 bp, Artificial Sequence
  Oligonucleotide nucleobase sequence motif
ccagggcagggagc

SEQ ID NO: 503 - DNA, 14 bp, Artificial Sequence
  Oligonucleotide nucleobase sequence motif
gcccagggcaggga

SEQ ID NO: 504 - DNA, 14 bp, Artificial Sequence
  Oligonucleotide nucleobase sequence motif
gtgcccagggcagg

SEQ ID NO: 505 - DNA, 14 bp, Artificial Sequence
  Oligonucleotide nucleobase sequence motif
gggtgcccagggca

SEQ ID NO: 506 - DNA, 14 bp, Artificial Sequence
  Oligonucleotide nucleobase sequence motif
gggggtgcccaggg

SEQ ID NO: 507 - DNA, 14 bp, Artificial Sequence
  Oligonucleotide nucleobase sequence motif
cagggggtgcccag

SEQ ID NO: 508 - DNA, 14 bp, Artificial Sequence
  Oligonucleotide nucleobase sequence motif
cgcagggggtgccc

SEQ ID NO: 509 - DNA, 14 bp, Artificial Sequence
  Oligonucleotide nucleobase sequence motif
ccaccgcagggggt

SEQ ID NO: 510 - DNA, 14 bp, Artificial Sequence
  Oligonucleotide nucleobase sequence motif
ggggccaccgcagg

SEQ ID NO: 511 - DNA, 14 bp, Artificial Sequence
  Oligonucleotide nucleobase sequence motif
tcggggccaccgca

SEQ ID NO: 512 - DNA, 14 bp, Artificial Sequence
  Oligonucleotide nucleobase sequence motif
cgtcggggccaccg

SEQ ID NO: 513 - DNA, 14 bp, Artificial Sequence
  Oligonucleotide nucleobase sequence motif
gtcgtcggggccac

SEQ ID NO: 514 - DNA, 14 bp, Artificial Sequence
  Oligonucleotide nucleobase sequence motif
ctgtcgtcggggcc

SEQ ID NO: 515 - DNA, 14 bp, Artificial Sequence
  Oligonucleotide nucleobase sequence motif
tcagctgtcgtcgg

SEQ ID NO: 516 - DNA, 14 bp, Artificial Sequence
  Oligonucleotide nucleobase sequence motif
cctcagctgtcgtc

SEQ ID NO: 517 - DNA, 14 bp, Artificial Sequence
  Oligonucleotide nucleobase sequence motif
ctcctcagctgtcg

SEQ ID NO: 518 - DNA, 14 bp, Artificial Sequence
  Oligonucleotide nucleobase sequence motif
cactcctcagctgt

SEQ ID NO: 519 - DNA, 14 bp, Artificial Sequence
  Oligonucleotide nucleobase sequence motif
gtcactcctcagct

SEQ ID NO: 520 - DNA, 14 bp, Artificial Sequence
  Oligonucleotide nucleobase sequence motif
tggtcactcctcag

SEQ ID NO: 521 - DNA, 14 bp, Artificial Sequence
  Oligonucleotide nucleobase sequence motif
tgtggtcactcctc

SEQ ID NO: 522 - DNA, 14 bp, Artificial Sequence
  Oligonucleotide nucleobase sequence motif
cttgtggtcactcc

SEQ ID NO: 523 - DNA, 14 bp, Artificial Sequence
  Oligonucleotide nucleobase sequence motif
accttgtggtcact

SEQ ID NO: 524 - DNA, 14 bp, Artificial Sequence
  Oligonucleotide nucleobase sequence motif
agaccttgtggtca

SEQ ID NO: 525 - DNA, 14 bp, Artificial Sequence
  Oligonucleotide nucleobase sequence motif
agagaccttgtggt

SEQ ID NO: 526 - DNA, 14 bp, Artificial Sequence
  Oligonucleotide nucleobase sequence motif
gcagagaccttgtg

SEQ ID NO: 527 - DNA, 14 bp, Artificial Sequence
  Oligonucleotide nucleobase sequence motif
gggcagagaccttg

SEQ ID NO: 528 - DNA, 14 bp, Artificial Sequence
  Oligonucleotide nucleobase sequence motif
gtgggcagagacct

SEQ ID NO: 529 - DNA, 14 bp, Artificial Sequence
  Oligonucleotide nucleobase sequence motif
ctgtgggcagagac

SEQ ID NO: 530 - DNA, 14 bp, Artificial Sequence
  Oligonucleotide nucleobase sequence motif
cactgtgggcagag

SEQ ID NO: 531 - DNA, 14 bp, Artificial Sequence
  Oligonucleotide nucleobase sequence motif
agcactgtgggcag

SEQ ID NO: 532 - DNA, 14 bp, Artificial Sequence
  Oligonucleotide nucleobase sequence motif
cgagcactgtgggc

SEQ ID NO: 533 - DNA, 14 bp, Artificial Sequence
  Oligonucleotide nucleobase sequence motif
cccgagcactgtgg

SEQ ID NO: 534 - DNA, 14 bp, Artificial Sequence
  Oligonucleotide nucleobase sequence motif
accccgagcactgt

SEQ ID NO: 535 - DNA, 14 bp, Artificial Sequence
  Oligonucleotide nucleobase sequence motif
gcaccccgagcact

SEQ ID NO: 536 - DNA, 14 bp, Artificial Sequence
  Oligonucleotide nucleobase sequence motif
ccgcaccccgagca

SEQ ID NO: 537 - DNA, 14 bp, Artificial Sequence
  Oligonucleotide nucleobase sequence motif
caccgcaccccgag

SEQ ID NO: 538 - DNA, 14 bp, Artificial Sequence
  Oligonucleotide nucleobase sequence motif
gacaccgcaccccg

SEQ ID NO: 539 - DNA, 14 bp, Artificial Sequence
  Oligonucleotide nucleobase sequence motif
cagacaccgcaccc

SEQ ID NO: 540 - DNA, 14 bp, Artificial Sequence
  Oligonucleotide nucleobase sequence motif
cccagacaccgcac

SEQ ID NO: 541 - DNA, 14 bp, Artificial Sequence
  Oligonucleotide nucleobase sequence motif
agcccagacaccgc

SEQ ID NO: 542 - DNA, 14 bp, Artificial Sequence
  Oligonucleotide nucleobase sequence motif
gcagcccagacacc

SEQ ID NO: 543 - DNA, 14 bp, Artificial Sequence
  Oligonucleotide nucleobase sequence motif
tcgcagcccagaca

SEQ ID NO: 544 - DNA, 14 bp, Artificial Sequence
  Oligonucleotide nucleobase sequence motif
cttcgcagcccaga

SEQ ID NO: 545 - DNA, 14 bp, Artificial Sequence
  Oligonucleotide nucleobase sequence motif
cacttcgcagccca

SEQ ID NO: 546 - DNA, 14 bp, Artificial Sequence
  Oligonucleotide nucleobase sequence motif
tccacttcgcagcc

SEQ ID NO: 547 - DNA, 14 bp, Artificial Sequence
  Oligonucleotide nucleobase sequence motif
gatccacttcgcag

SEQ ID NO: 548 - DNA, 14 bp, Artificial Sequence
  Oligonucleotide nucleobase sequence motif
gggatccacttcgc

SEQ ID NO: 549 - DNA, 14 bp, Artificial Sequence
  Oligonucleotide nucleobase sequence motif
gggggatccacttc

SEQ ID NO: 550 - DNA, 14 bp, Artificial Sequence
  Oligonucleotide nucleobase sequence motif
aagggggatccact

SEQ ID NO: 551 - DNA, 14 bp, Artificial Sequence
  Oligonucleotide nucleobase sequence motif
gaaagggggatcca

SEQ ID NO: 552 - DNA, 14 bp, Artificial Sequence
  Oligonucleotide nucleobase sequence motif
aagaaagggggatc

SEQ ID NO: 553 - DNA, 14 bp, Artificial Sequence
  Oligonucleotide nucleobase sequence motif
ccaagaaaggggga

SEQ ID NO: 554 - DNA, 14 bp, Artificial Sequence
  Oligonucleotide nucleobase sequence motif
gcccaagaaagggg

SEQ ID NO: 555 - DNA, 14 bp, Artificial Sequence
  Oligonucleotide nucleobase sequence motif
gtgcccaagaaagg

SEQ ID NO: 556 - DNA, 14 bp, Artificial Sequence
  Oligonucleotide nucleobase sequence motif
cagtgcccaagaaa

SEQ ID NO: 557 - DNA, 14 bp, Artificial Sequence
  Oligonucleotide nucleobase sequence motif
tgcagtgcccaaga

SEQ ID NO: 558 - DNA, 14 bp, Artificial Sequence
  Oligonucleotide nucleobase sequence motif
gctgcagtgcccaa

SEQ ID NO: 559 - DNA, 20 bp, homo sapies
actggacccatgtggggatc

SEQ ID NO: 560 - DNA, 32 bp, homo sapiens
gcacgactctcctttccaggctgccttgccgg

SEQ ID NO: 561 - DNA, 32 bp, homo sapiens
aggctgccttgccggcggggggagcccgaaac

SEQ ID NO: 562 - DNA, 37 bp, homo sapiens
ggggggagcccgaaacagccctttgcagctctgcacc

SEQ ID NO: 563 - DNA, 30 bp, homo sapiens
cctttgcagctctgcaccaggagcaggttt

SEQ ID NO: 564 - DNA, 23 bp, homo sapiens
aggttttgcggaacccccatgta

SEQ ID NO: 565 - DNA, 26 bp, homo sapiens
ggaacccccatgtaaggcttccccgg